(19) United States
(12) Patent Application Publication
(10) Pub. No.: US 2026/0207921 A1
(43) Pub. Date: Jul. 23, 2026

(54) APPARATUS AND METHODS FOR OPTIMIZING INTRA CARDIAC FILLING PRESSURES, HEART RATE, AND CARDIAC OUTPUT

(71) Applicant: Ablation Innovations, LLC, Brentwood, TN (US)

(72) Inventors: Daniel Walter Kaiser, Nashville, TN (US); Margaret Rox, Brentwood, TN (US)

(21) Appl. No.: 19/452,750

(22) Filed: Jan. 19, 2026

Related U.S. Application Data
(63) Continuation-in-part of application No. PCT/US24/38894, filed on Jul. 19, 2024.
(60) Provisional application No. 63/527,710, filed on Jul. 19, 2023.
(60) Provisional application No. 63/532,884, filed on Aug. 15, 2023.

(51) Int. Cl. A61M 60/295; A61M 60/13; A61M 60/135; A61M 60/157; A61M 60/30; A61M 60/468; A61M 60/497; A61M 60/531; A61M 60/569; A61M 60/843; A61M 60/886; A61M 60/90

(52) U.S. Cl. CPC A61M 60/295; A61M 60/13; A61M 60/135; A61M 60/157; A61M 60/30; A61M 60/468; A61M 60/497; A61M 60/531; A61M 60/569; A61M 60/843; A61M 60/886; A61M 60/90; A61M 2202/0007; A61M 2202/0014; A61M 2202/0208; A61M 2202/0225; A61M 2205/04; A61M 2205/054; A61M 2205/3303; A61M 2205/3327; A61M 2205/3344; A61M 2205/50; A61M 2210/125; A61M 2230/005; A61M 2230/04; A61M 2230/20

(57) ABSTRACT

Apparatus, systems, and methods are provided for optimizing intracardiac filling pressures and cardiac output in patients with heart failure, conduction disease, and atrial fibrillation. The system is able to adjust and optimize intracardiac filling pressures and cardiac output by adjusting heart rate and the effective amount of total body blood volume. The device includes an adjustable member that may create a mean pressure differential in order to manifest an effective “mechanical diuresis” by sequestering extraneous blood volume to the high-capacitance of the venous vasculature. The system is therefore designed to reduce intracardiac filling pressures while maintaining or even increasing cardiac output.

### RELATED APPLICATION DATA

The present application is a continuation of co-pending International Application No. PCT/US2024/038894, filed Jul. 19, 2024, which claims benefit of and priority to provisional applications Ser. Nos. 63/527,710, filed Jul. 19, 2023, and 63/532,884, filed Aug. 15, 2023, the entire disclosures of which are expressly incorporated by reference herein. The present application is also related to application Ser. No. 17/372,487, filed Jul. 11, 2021, which is a continuation of Ser. No. 15/882,887, filed Jan. 29, 2018, now U.S. Pat. No. 11,058,962, which is a continuation of application Ser. No. 14/597,190, filed Jan. 14, 2015, now U.S. Pat. No. 9,878,080, which claims benefit of provisional application Ser. No. 61/927,038, filed Jan. 14, 2014, the entire disclosures of which are expressly incorporated by reference herein.

### TECHNICAL FIELD

The present application relates to apparatus, systems, and methods for prevention and/or remediation of heart disease, e.g., for optimizing intra-cardiac filling pressures, heart rate, and/or cardiac output for patients, such as patients suffering from conduction disease, atrial fibrillation, and/or congestive heart failure.

### BACKGROUND

Placing a permanent pacemaker is often the only effective treatment for patients with sick sinus syndrome, atrioventricular conduction disorders, and chronotropic incompetence (where the heart rate does not increase in proportion to the demands of the body). In these circumstances, patients may receive dual chamber pacemakers, e.g., with one pacing lead typically placed in the right atrium (RA) and a second endocardial pacing lead typically placed in the right ventricular (RV) apex. However, RV pacing is associated with increased rate of death or hospitalization from congestive heart failure. The pathophysiology for this is unclear, but growing evidence suggests that an associated increase in intra-cardiac filling pressures is the primary driver of poor outcomes. RV pacing may cause bi-ventricular dyssynchrony, which, in attempts to maintain adequate cardiac output, may result in a new equilibrium of higher intra-cardiac filling pressures, particularly left-sided filling pressures. Higher intra-cardiac pressures result in symptoms of shortness of breath, an increase in the likelihood of cardiac arrhythmias, and a decrease in exercise capacity. The only current alternative in these patients is to place bi-ventricular pacing leads, whereby a third pacing lead is placed typically into the coronary sinus of the heart in order to pace both ventricles. This results in simultaneous bi-ventricular contraction, also referred to as cardiac resynchronization therapy (CRT). CRT is associated with lower left-sided filling pressures, decreased arrhythmias, decreased heart failure hospitalizations, and decreased mortality. However, approximately ten percent (10%) of patients cannot receive CRT due to heart anatomy. Therefore, an alternative device that is able to pace the heart while maintaining lower intra-cardiac filling pressures would be advantageous.

Heart failure occurs because the body perceives a decrease in cardiac output. The normal homeostasis feedback mechanisms increase total body volume in efforts to increase stroke volume in order to maintain cardiac output by the Frank-Starling mechanism. However, as cardiac performance continues to decline, the ability of the Frank-Starling mechanism is exceeded, and an increasing total body volume continues despite no improvement in stroke volume. The body continues this volume accumulation (via fluid retention), which ultimately worsens cardiac performance and leads to congestive heart failure. In this relatively common situation, a reduction in total body volume via diuretics (against the normal feedback mechanisms) may improve cardiac function and the patient's symptoms.

Heart failure costs over thirty billion dollars annually and is expected to grow by over two hundred percent (200%) over the next twenty years. Heart failure is the leading cause of hospitalization in people over the age of sixty-five and accounts for one-fifth of all elderly admission. Despite advances in monitoring and therapies, the thirty-day readmission rate for congestive heart failure has remained extremely high and relatively unchanged (slightly over 20%) over the last two decades in the United States. In heart failure patients, increasing the heart rate may increase the cardiac output and help with diuresis. However, higher heart rates are associated with higher intracardiac filling pressures, which limit patient exercise capacity. A method to increase cardiac output without increasing intracardiac pressures would be advantageous.

Heart failure results in increased fluid retention. The increased fluid causes morbidity in a variety of ways. Increased fluid in the extremities can lead to edema and tissue congestion, increased fluid in the liver can lead to cirrhosis and liver failure, and increased fluid in the gastrointestinal track can decrease food and drug absorption, and lead to early satiety. Increased venous pressure decreases renal perfusion pressure, which can lead to renal dysfunction and more fluid retention. However, the primary patient symptom associated with admission to the hospital is related to elevated left ventricular filling pressures. Elevated left ventricular diastolic filling pressure backs up into the left atrium and the pulmonary veins. An elevation of pulmonary venous pressure can cause tachypnea, increase respiratory dead space, and worsen oxygen exchange. In addition, elevated left-sided filling pressures can cause pulmonary edema and decreased oxygen exchange which results in shortness of breath, orthopnea (shortness of breath when laying down), and hypoxia (decreased oxygen levels).

The kidneys play a crucial role in regulating fluid balance, blood pressure, and waste elimination in the body. Proper renal function depends on an effective blood flow through the renal arteries and veins. The renal veins are responsible for draining deoxygenated blood from the kidneys back to the heart via the inferior vena cava (IVC). When renal venous flow is impaired, it can lead to a buildup of pressure within the kidneys, causing a condition known as renal congestion.

Elevated pressure in the renal veins can have several detrimental effects, including renal congestion, where increased venous pressure can lead to stasis of blood within the kidneys, reducing their ability to filter blood effectively. This can result in reduced glomerular filtration rate (GFR), impaired urine output, and accumulation of toxins in the body. Additional effects include renal impairment, where elevated renal vein pressure can cause damage to the delicate structures of the kidneys, leading to renal impairment or failure. This is particularly concerning in patients with pre-existing conditions such as heart failure, where renal function is already compromised. Another potential effect is fluid retention, where impaired renal function due to elevated venous pressure leads to sodium and water retention, exacerbating symptoms of heart failure such as edema, ascites, and pulmonary congestion.

Patients with heart failure often suffer from elevated central venous pressure (CVP), which is transmitted back to the renal veins, exacerbating renal congestion and impairment. This creates a vicious cycle where worsening heart function leads to deteriorating kidney function, further exacerbating heart failure symptoms—a condition known as cardiorenal syndrome.

Intubated patients, particularly those undergoing high-risk surgeries, are at an increased risk of renal congestion and impairment. Mechanical ventilation can increase intrathoracic pressure, which in turn raises CVP and consequently renal vein pressure. This elevated pressure can lead to significant renal dysfunction in these patients, complicating their postoperative recovery and increasing the risk of acute kidney injury (AKI). Complications include mechanical ventilation, where positive pressure ventilation increases intrathoracic pressure, which can be transmitted to the venous system, leading to elevated CVP and renal vein pressure; hemodynamic instability, as high-risk surgeries often involve significant hemodynamic fluctuations, which can compromise renal perfusion and exacerbate venous congestion; and increased mortality and morbidity as renal impairment in intubated and high-risk surgical patients is associated with higher rates of morbidity and mortality, prolonged hospital stays, and increased healthcare costs.

There is a critical need for innovative solutions that can mechanically enhance renal venous flow, reduce renal vein pressure, and improve renal function in patients with heart failure, those undergoing high-risk surgeries, and intubated patients. These solutions should be capable of: Creating Low-Pressure Zones—mechanically creating areas of low pressure within the IVC to facilitate renal venous drainage; Real-Time Monitoring and Adjustment—integrating sensors to monitor venous pressures and adjust the device's operation dynamically; Minimally Invasive Implementation—ensuring the device can be implanted or deployed with minimal invasiveness to reduce patient risk and recovery time; and Improving Patient Outcomes—ultimately enhancing renal function, reducing fluid retention, and improving overall patient outcomes, particularly in vulnerable populations such as heart failure patients and those undergoing high-risk surgical procedures.

Elevated renal vein pressure and impaired renal venous flow are significant concerns in patients with heart failure, particularly those who are intubated or undergoing high-risk surgeries. These conditions exacerbate renal impairment, fluid retention, and overall morbidity and mortality. There is a pressing need for mechanical devices and methods that can effectively reduce renal vein pressure, enhance venous return, and improve renal function. Such innovations have the potential to significantly improve patient outcomes and reduce healthcare burdens associated with renal and heart failure complications.

### SUMMARY

The present application is directed to an apparatus, systems, and methods for prevention and/or remediation of heart disease, e.g., for optimizing intra-cardiac pressures, heart rate, and cardiac output for patients, such as patients suffering from conduction disease, atrial fibrillation, and congestive heart failure. More particularly, the present application is directed to implantable devices that measure hemodynamics and help optimize intra-cardiac pressures, heart rate, and/or cardiac output for patients; and systems and methods for using such devices. The present application also relates to medical devices and methods for enhancing renal venous flow, reducing renal vein pressure, and improving patient outcomes in conditions such as heart failure, chronic kidney disease, and venous congestion, especially in high-risk surgical patients and those requiring intubation.

The primary treatment for patients with heart failure is to give diuretic medications to reduce total body volume, with more refractory cases requiring continuous infusion of medications to improve cardiac function. Reducing total body fluid results in less congestion, improved renal function, and a paradoxical increase in cardiac output. A device that is able to induce “mechanical diuresis” where excess fluid is sequestered elsewhere in a patient's body may be able to optimize intracardiac pressures and cardiac output similarly to diuretics. We have demonstrated that by creating a pressure gradient in the inferior vena cava, we can move extraneous and congesting fluid to the high capacitance vessels below a pressure gradient device placed within or downstream of the inferior vena cava. Given the high capacitance of the venous system, a large volume of blood can be relocated, with a significant decrease in intra-cardiac pressures and with only a minimal (if any) increase in pressure below our device.

Computer modeling has revealed that differences in bi-ventricular function are likely more important than total cardiac output in terms of patient symptoms. However discordant the ventricular function may be, the cardiac output of each ventricle must be the same. Therefore, with predominant left heart failure (or a hyperactive right ventricle), pressure builds behind the left ventricle. In predominant right heart failure (or a hyperactive left ventricle), pressure builds behind the right ventricle. Of importance, exercise/exertion exaggerates differences in ventricular function. Over time and accelerated by disease, most left ventricles develop some degree of diastolic dysfunction. Accordingly, during exercise, the left ventricle cannot “keep up with” the right ventricle. The resulting equilibrium reveals rapid elevations in left-sided filling pressures during exercise that limit exercise capacity. An interactive device that can rapidly sequester extra blood volume can limit the rise in intra-cardiac filling pressures while maintaining cardiac output would dramatically improve exercise capacity and improve patient symptoms of fatigue and shortness of breath.

Long standing elevated left atrial (LA) pressure can dilate the left atrium and contribute to atrial arrhythmias such as atrial fibrillation. It is likely that increases in ventricular pressures (both right and left ventricular pressures) may result in ventricular arrhythmias as well. By maintaining lower intracardiac filling pressures, a system that maintains cardiac output while preventing elevated intracardiac pressures may decrease deadly cardiac arrhythmias.

Cardiac output and heart rate have a complicated relationship. In patients without volume overload, increasing heart rate does not affect cardiac output. This apparent paradox occurs due to a decrease in ventricular filling time and a redistribution of blood from the heart to peripheral tissue. When venous return to the heart is not increased, the stroke volume will fall with increasing heart rate implying an extrathoracic venous collapse. However, in patients that are volume overloaded, increasing the heart rate can maintain stroke volume and increase cardiac output dramatically. The amount of improvement in cardiac output typically depends on the ability of the left ventricle to relax fast enough at higher heart rates. Therefore, optimizing cardiac output is a complex interaction between volume status, heart rate, bi-ventricular pressure-volume curves and filling parameters. Improving heart rate may increase left-sided filling pressures and exacerbate patient symptoms. Left-sided filling pressures may rise significantly with only marginal improvement in cardiac output. Therefore, the relationships between cardiac output in response to changes in left-sided filling pressures are an important consideration to optimize filling pressures and cardiac output.

In most individuals, cardiac output does not need to be increased. However, in patients with heart failure, the body senses a reduction in cardiac output. In these patients, increasing cardiac output can improve renal perfusion and urine output, thereby improving volume status. In order to most effectively titrate heart rate, the systems and methods herein may monitor both intracardiac pressures and cardiac output.

Most pacemaker systems adjust heart rate based on physiologic need (such as monitoring respiratory rate and/or patient activity) as opposed to adjusting heart rate based on intracardiac pressures, cardiac output, and total body blood volume. In most patients with volume overload, increasing the heart rate will increase the cardiac output. However, this often results in elevated left-sided filling pressures and worsening symptoms of heart failure. Therefore, while pacing improves cardiac output and volume status, it may make symptoms of heart failure worse. If the intra-cardiac pressures can be monitored and optimized with increasing heart rates, total body volume can be improved without exacerbating heart failure symptoms. Furthermore, by adjusting both heart rate and intra-cardiac filling pressures, cardiac output can be optimized. This encompasses a novel concept—to adjust cardiac output based on a measure of patient volume status in order to improve symptoms and encourage diuresis, as needed. Therefore, if a patient begins to gain excess blood volume, the systems and methods herein may increase cardiac output in efforts to diurese the excess blood physiologically.

Prior systems attempting to optimize left-sided filling pressures suggest placing a pressure sensor directly into the left atrium or left ventricle. However, such sensors are at risk of thrombus development, which may lead to thromboembolic events. By not interfering with the systemic circulation or integrity of natural barriers (such as the interatrial septum), thrombus development at the location of pressure sensors is prevented from causing systemic thromboembolic events by utilizing the ability of the lung vasculature to sequester these thrombi from reaching the brain and other key organs. Furthermore, placing a pressure sensor directly into the coronary sinus, with or without an occlusion, the pressure of the left ventricle may be estimated.

Additionally, monitoring changes in right ventricular or pulmonary pressures—or combining right ventricular or pulmonary pressures with an estimate of stroke volume—may be used to estimate left-sided filling pressures without requiring a sensor in the left atrium. Mild changes in the pressure gradient in the inferior vena cava, for example, may cause slight changes in right-sided filling pressures and subsequent changes in stroke volume by the Frank-Starling mechanism. However, if the left ventricle is unable to increase the stroke volume similarly, the pressure of the left ventricle, left atrium, pulmonary artery, and right ventricle increases. This increase in afterload decreases right ventricular stroke volume until it equals the stroke volume of the left ventricle. Therefore, when mild changes in the pressure gradient mechanism cause a significant increase in right or left sided pressures, this suggests the additional volume did not improve cardiac output. Therefore, only changes in pressure (or changes in stroke volume) are necessary to estimate the pressure differential required to maximize cardiac output.

Additionally, measuring changes in pressure may be used to gain an accurate measure of cardiac output by utilizing a Venturi-meter effect and a form of Poiuselle's Law, which can be used to infer that changes in pressure are proportional to a relationship between the flow rate and the radius of the vein. Such changes may be used to determine flow rate (cardiac output) by directly perturbing the pressure differential in the vein and collecting multiple measurements at different levels of venous occlusion. Thus, the system can be used to measure pressures and cardiac output, as well as inducing pressure changes to reduce total blood volume.

In accordance with one example, the apparatus, systems, and methods herein may determine an elevation in total body blood volume and optimize cardiac output by adjusting heart rate in efforts to improve volume status through increased renal perfusion and therefore increased diuresis. In addition, the apparatus and systems herein may control the intra-cardiac filling pressures by creating a pressure differential in a vessel such as the inferior vena cava. The pressure differential may sequester extraneous blood volume to the high capacitance of the venous system. The apparatus and systems herein may manifest an effective “mechanical diuresis” to improve myocardial hemodynamics. Optionally, the apparatus, systems, and methods herein may also pace the right ventricle in patients requiring pacing, and/or simultaneously may prevent the rise in intra-cardiac pressures associated with right ventricular pacing to improve the symptoms and outcomes of patients. Therefore, the apparatus and systems herein may be an alternative to placing a pacing lead in the coronary sinus (bi-ventricular pacing), which is the primary treatment option for most patients requiring right ventricular pacing and may be the only treatment option for up to ten percent (10%) of patients that have inadequate coronary anatomy for the additional pacing lead.

Optionally, the systems and methods herein may also communicate to the patient their total body blood volume status and/or help manage their medical management. In addition or alternatively, the apparatus, systems, and methods may be responsive to patient activity to optimize cardiac performance and intracardiac pressures to improve exercise capacity. Furthermore, decreasing intra-cardiac pressures may reduce ectopic beats, thereby reducing dangerous and symptomatic arrhythmias. In addition, reducing intracardiac pressures may result in remodeling that improves myocardial function and hemodynamics. Thus, the apparatus, systems, and methods herein may therefore improve patient respiratory symptoms, improve cardiac output and/or encourage diuresis to reduce total body volume, improve exercise capacity, guide patient medication dosing, prevent atrial and ventricular arrhythmias, prevent negative heart failure remodeling, decrease heart failure hospitalization rates, and/or improve the quality and duration of life.

In accordance with another example, the apparatus, systems, and methods herein may include implantation of a device that monitors pressures in and around the heart, as well as the cardiac output, and accordingly adjusts heart rate and a pressure gradient, such as through the inferior vena cava. The pressure gradient and heart rate may be adjusted to optimize the filling pressures inside of the heart and the cardiac output. In response to measurements of cardiac output and/or sensors of pressure measurements, at least one pressure gradient and/or the heart rate are adjusted to optimize hemodynamics.

Monitoring intracardiac filling pressure may include one or more pressure sensors located in the coronary sinus, pulmonary circulation, right ventricle, right atrium, left atrium, and/or vena cava. In addition or alternatively, monitoring stroke volume or cardiac output may be determined by assessing for changes in electrical impedance or changes in oxygen saturation, such as, determined by light oximetry.

The systems and methods herein expand on existing apparatus, systems, and methods for optimizing intracardiac filling pressures and cardiac output in patients with heart failure, conduction disease, and atrial fibrillation. In addition to mechanical diuresis and heart rate modulation, this enhanced system incorporates a multi-faceted approach to achieve comprehensive cardiac management.

Renal denervation, a procedure that disrupts sympathetic nerve signaling to the kidneys, may be integrated as an adjunct therapy. By reducing renal sodium and water reabsorption, it can complement the mechanical diuresis achieved by the pressure differential device, leading to further reduction in fluid overload and improved cardiac function.

Moreover, the system may incorporate continuous, real-time monitoring of intracardiac pressures, cardiac output, and other relevant biomarkers. This continuous stream of physiological data enables the system to operate in a closed-loop manner, automatically adjusting therapy parameters based on individual patient responses.

In accordance with still another example, systems and methods may be provided for determining optimal estimated left sided filling pressure within a patient's heart. The system and methods may gradually adjust the pressure differential and/or heart rate in order to create pressure-cardiac output relationships. The systems and methods may periodically be gradually adjusted to alter the pressure differential, which may result in a new estimated left sided filling pressure and a measured cardiac output, which may be recorded. Optionally, the heart rate may also periodically be gradually adjusted, which may result in a new estimated left sided filling pressure and measured cardiac output which can be recorded. Therefore, by altering the pressure differential and heart rate, the systems and methods may maximize cardiac output with respect to intracardiac filling pressures. Pressure-cardiac output relationships may be determined in order to identify the optimal pressure differential and heart rate in order to optimize cardiac output at an acceptable rise in the estimated left sided filling pressures at various patient activity levels.

In another example, systems and methods may be provided to create a pressure differential that include one or more shunts, balloons, orifices, valves, constriction mechanism, and the like, e.g., to increase a pressure drop through the tricuspid valve, superior vena cava, inferior vena cava, pulmonary artery, and/or right atrium.

In accordance with an example, a system is provided for implantation in the body of a patient with conduction disease and/or heart failure configured to monitor and/or treat the patient. The system may include at least one sensor configured to provide sensor data corresponding to pressures within or near the heart; at least one adjustable component configured to create a pressure gradient to blood flow; at least one pacing component configured to at least one of sense and pace the heart; and a controller configured for adjusting the function of at least one component based at least in part on sensor data from at least one sensor.

Inflatable balloons can be positioned at various locations within the venous system to create occlusions, helping to manage blood flow and reduce venous pressure. Self-expanding stents made from shape-memory materials like Nitinol can be used for occlusion, providing a robust and adaptive method to control blood flow. Expandable anchoring legs, similar to those on IVC filters, can be used to secure devices within vessels, preventing migration and ensuring stability. Helical devices or screws can be rotated to anchor within vessel walls, providing a mechanical means to secure the device in place. Magnetic anchoring can use internal and external magnets to secure devices, offering a non-invasive method to maintain device position. Temporary adhesives can adhere occlusion devices to vessel walls, providing a secure yet temporary fixation. Anchoring wires or tethers can extend to engage with vessel walls or bifurcations for secure positioning, ensuring the device remains in place during use. Devices with textured or high-friction surfaces can prevent movement once positioned, providing stability without additional fixation mechanisms. Mechanical expandable members can be actuated to change shape and size for occlusion within vessels, offering a flexible and adjustable method to control blood flow. Various types of valves, such as ball, butterfly, diaphragm, gate, and globe valves, can be used to control blood flow, providing precise and adjustable occlusion. Propeller mechanisms can adjust the cross-sectional area of blood flow for occlusion, offering dynamic control over blood flow rates. Suction mechanisms can create localized low-pressure zones to aid in blood flow control, providing an effective method to manage venous return. Miniaturized impellers can be used as rotationally sensitive elements for blood flow control, offering a mechanical means to influence blood flow within vessels.

In accordance with another example, a system is provided that creates a partial obstruction both above and below the renal veins within the inferior vena cava (IVC). This design is useful for generating a low-pressure zone, which facilitates the movement of blood from the renal veins towards the heart. In this system, a pumping balloon is strategically placed between two occlusion balloons. The lower balloon, located below the renal veins, creates a partial or complete occlusion. The upper balloon, situated above the pumping balloon, provides a downstream obstruction. The synchronized inflation and deflation of these balloons generate a peristaltic effect, ensuring efficient blood flow and maintaining a negative pressure area at the level of the renal veins. This configuration is essential because the pumping balloon alone cannot create the necessary low-pressure zone without the upstream and downstream occlusions, which work together to enhance venous return.

In some examples, the proximal balloon positioned below the renal veins is designed to achieve complete occlusion of the IVC. While it may be intuitive to assume that complete occlusion would negatively impact cardiac output, clinical studies have demonstrated otherwise, particularly in patients with heart failure. These studies suggest that complete occlusion below the renal veins for durations of 5-10 minutes or potentially longer can effectively manage venous pressures without causing significant drops in cardiac output. This approach contrasts with previous assumptions and highlights a novel method for reducing renal vein pressure and improving renal function in heart failure patients.

In some examples, the distal balloon is positioned higher up in the IVC. This balloon serves a dual purpose: it occludes the IVC to maintain the negative pressure zone at the renal veins and acts as a diagnostic tool to measure central venous pressure (CVP) and cardiac output. By occluding the IVC at a higher level, the distal balloon helps to lower CVP, thereby reducing the filling pressures on the heart. Additionally, integrated sensors within this balloon provide real-time data on hemodynamic parameters, enabling precise monitoring and adjustment of the device's operation to optimize patient outcomes.

In some examples, accurate measurement of cardiac output is an important component of the systems and methods herein. Various methods can be employed to achieve this, including the use of impedance catheters placed in the right ventricle. These catheters measure changes in electrical impedance that correlate with blood flow, providing continuous cardiac output data. Another method involves measuring the pressure drop across a partially occluded segment of the IVC, using the delta pressure drop concept. This technique involves creating a controlled partial obstruction and measuring the resulting pressure changes to infer flow rates and cardiac output.

In some examples, the system includes a balloon catheter that can be advanced into the pulmonary artery, similar to a Swan-Ganz catheter. This addition allows for comprehensive monitoring of pulmonary artery pressures and cardiac output. The catheter can provide valuable data on right heart function and pulmonary hemodynamics, enhancing the overall effectiveness of the system in managing heart failure and related conditions.

The system may also feature a smaller balloon catheter with integrated impedance sensors that can travel beyond the primary venous balloon pump and into the pulmonary artery. This catheter includes a mechanism to slide and lock it in place, ensuring stable and accurate placement. The impedance sensors provide detailed measurements of blood flow and cardiac output, offering a comprehensive view of the patient's hemodynamic status and enabling precise adjustments to therapy.

In some examples, the catheter can be designed to incorporate oxygen delivery mechanisms. This can be particularly useful in patients who require supplemental oxygen due to respiratory insufficiency or during procedures where maintaining adequate oxygenation is critical. The oxygen delivery system can be integrated within the catheter, allowing for the simultaneous measurement of hemodynamic parameters and administration of oxygen.

In other examples, the device is an elongated catheter, incorporating a multitude of balloons and sensors strategically placed along its length. These components work in tandem to create pressure gradients and measure various hemodynamic parameters, including blood flow and oxygen saturation. By leveraging a modified Poiseuille's equation, the system can accurately assess flow dynamics in response to balloon inflation, providing critical insights into the patient's cardiovascular status.

The device can selectively inflate and deflate multiple balloons incorporated along the catheter. By analyzing the pressure changes that occur during these manipulations, the device can calculate blood flow through the partially occluded section of the vena cava. This real-time data is essential for understanding the patient's hemodynamic status and for making informed clinical decisions.

In addition to its pressure and flow measurement capabilities, the device may be equipped with a variety of sensors designed to monitor other key hemodynamic parameters. Oxygen saturation sensors are integrated along the catheter to provide continuous monitoring of blood oxygen levels at different locations. This feature is crucial for estimating regional oxygen consumption, which can help in identifying areas of tissue ischemia or metabolic distress. For instance, a significant drop in oxygen saturation in a specific region could indicate the presence of ischemic gut, a condition that requires immediate medical attention.

The system's ability to measure and analyze hemodynamic parameters extends to the integration of additional sensors, such as temperature, pH, biochemical, and electrochemical sensors. These sensors enhance the device's capability to monitor a comprehensive range of physiological conditions. Temperature sensors help in assessing metabolic rates and detecting hyperthermia or hypothermia. pH sensors provide insights into the acid-base balance, which is crucial in managing patients with metabolic or respiratory disorders. Biochemical sensors, capable of detecting lactate and glucose levels, offer valuable data on tissue perfusion and metabolic activity. Electrochemical sensors monitor electrolyte levels, ensuring the maintenance of cardiac and neural functions.

Furthermore, the device's advanced data collection capabilities, coupled with machine learning algorithms, enable real-time analysis and diagnostic support. By continuously collecting and processing data, the system can identify patterns and anomalies that may indicate underlying health issues. For example, changes in oxygen consumption patterns can be analyzed to detect early signs of organ dysfunction or infection sources. This predictive capability is particularly beneficial in critical care settings, where early intervention can significantly improve patient outcomes.

The device also offers substantial benefits in optimizing fluid resuscitation and managing cardiac output. By mapping the patient's Frank-Starling curve, the system can determine the optimal filling pressures that maximize cardiac output. This personalized hemodynamic management is essential in guiding fluid therapy, preventing fluid overload, and improving patient stability. Additionally, the partial occlusion feature of the device allows for strategic blood volume redistribution, which can enhance kidney function and reduce the risk of respiratory complications.

In another example, the system can function as a device to reduce renal vein blood pressure. This system can be inserted via a minimally invasive procedure through a peripheral vein, either in the leg or neck, and positioned strategically around the renal veins.

The system can comprise three main components: Distal Occlusion Segment: This segment is designed to isolate a distal portion of the IVC, creating a controlled environment for the device to operate; Proximal Occlusion Segment: Positioned proximally within the IVC, this segment works in tandem with the distal segment to isolate the target section of the IVC around the renal veins; Middle Balloon Segment: Located between the distal and proximal occlusion segments, this balloon inflates and deflates rhythmically to facilitate venous return and reduce renal vein pressure.

To ensure optimal performance and patient safety, the system incorporates multiple sensors, including but not limited to: Renal Vein Pressure Sensor: This sensor monitors the blood pressure at the location of the renal veins, providing real-time data to adjust the device's operation; Right Atrium Pressure Sensor: This additional sensor measures the pressure at the level of the right atrium, offering a comprehensive understanding of the patient's venous pressure dynamics.

A sophisticated processor receives data from both sensors, enabling precise control over the inflation and deflation of the middle balloon. This real-time adjustment mechanism ensures that the device can dynamically respond to changes in blood pressure, optimizing renal vein pressure reduction and enhancing overall cardiovascular function.

The method for reducing renal vein pressure using the venous balloon pump system involves the following steps. Insertion: The venous balloon pump system is inserted through a peripheral vein, typically accessed via the leg or neck, using minimally invasive techniques to ensure patient comfort and rapid recovery. Positioning: The distal and proximal occlusion segments are carefully positioned to isolate a section of the IVC around the renal veins. This placement is critical for creating a controlled environment in which the middle balloon can operate effectively. Monitoring Renal Vein Pressure: Using a dedicated sensor, the system continuously monitors blood pressure at the location of the renal veins. This data is crucial for adjusting the device's operation to achieve optimal pressure reduction. Monitoring Right Atrium Pressure: An additional sensor monitors pressure at the level of the right atrium. This information provides a comprehensive view of the patient's venous pressure, ensuring the device's adjustments are well-informed and effective. Data Processing and Control: The system's processor receives real-time data from both sensors. Based on this information, the processor controls the inflation and deflation of the middle balloon segment, ensuring precise regulation of blood flow and pressure. Inflation and Deflation: The middle balloon inflates and deflates rhythmically to facilitate venous return and reduce renal vein pressure. This action is dynamically adjusted based on the monitored blood pressures, ensuring the device responds appropriately to the patient's physiological needs.

In addition, the distal end of the system may include a distal opening that enables a smaller balloon catheter to be advanced through the device into the right atrium (RA), right ventricle (RV), and further into the pulmonary artery. This elongated member is capable of measuring pulmonary pressures, providing critical data to optimize the overall cardiovascular function of the patient.

This smaller balloon catheter is advanced through a distal opening in the distal occlusion segment, traversing the right atrium and right ventricle, and positioned within the pulmonary artery. The primary function of this balloon is to enhance RV stroke volume by synchronizing its inflation and deflation with the RV contraction.

The system employs an electrocardiogram (ECG) sensor to continuously monitor the patient's cardiac electrical signals. By analyzing the ECG waveform, the processor can accurately identify the phases of the cardiac cycle, specifically pinpointing the moments of RV contraction. When the ECG indicates that the RV is in systole (contraction phase), the processor triggers the pulmonary artery balloon to inflate, thereby aiding the propulsion of blood into the pulmonary artery. Conversely, during diastole (relaxation phase), the balloon deflates, allowing the RV to fill with blood for the next cycle. This synchronized action effectively boosts RV stroke volume, enhances cardiac output, and improves overall circulatory efficiency.

The system's processor plays a pivotal role in regulating the balloon's pumping frequency. It incorporates real-time data on the patient's heart rate and respiratory rate to dynamically adjust the inflation and deflation cycles. By integrating heart rate data, the processor ensures that the balloon inflations are synchronized with the cardiac cycle, optimizing the timing of each inflation to coincide with venous return phases, thus enhancing the efficiency of blood flow and pressure modulation.

Additionally, the processor considers the respiratory rate, as breathing significantly influences venous return and intrathoracic pressures. By aligning the balloon's pumping actions with the respiratory cycle, the system can further enhance hemodynamic stability. For instance, inflating the balloon during the inspiratory phase can capitalize on the increased venous return associated with negative intrathoracic pressure, while deflation during expiration can prevent excessive pressure buildup.

In septic shock, maintaining optimal fluid balance is critical to ensure adequate tissue perfusion without causing fluid overload. The venous balloon pump system, with its distal, proximal, and middle balloon segments, can dynamically adjust venous return by inflating and deflating the middle balloon segment based on real-time pressure data from the sensors located at the renal veins and right atrium. This helps to precisely control central venous pressure (CVP), guiding fluid resuscitation efforts. The processor analyzes the pressure data and adjusts the middle balloon's action to either increase or decrease venous return, ensuring that the patient maintains an optimal intravascular volume status.

For patients who require diuretic therapy to manage fluid overload, the venous balloon pump system provides valuable data on kidney perfusion through sensors monitoring renal vein pressure and oxygen levels. By continuously assessing these parameters, the system can indicate the patient's response to diuretics. If the sensors detect adequate renal perfusion and oxygenation, it suggests that diuretic therapy is effective. Conversely, if renal perfusion decreases, it may indicate a need to adjust the diuretic dose or change the therapeutic approach to prevent renal dysfunction.

The integrated oxygen sensors within the venous balloon pump system are strategically placed to monitor oxygenation levels in various organs, such as the liver, gut, kidneys, and myocardium. In septic shock, early detection of ischemia is crucial for timely intervention. The system can identify areas of low oxygenation, signaling potential ischemia in organs like the gut (suggesting mesenteric ischemia) or liver (indicating hepatic dysfunction). Continuous monitoring allows healthcare providers to detect these issues early, enabling prompt and targeted interventions to restore adequate perfusion and prevent organ failure.

In managing septic shock, the administration of vasopressors is often necessary to maintain adequate blood pressure and perfusion. The venous balloon pump system can guide the titration of vasopressors by providing real-time data on systemic pressures and oxygenation. By monitoring these parameters, the system helps optimize vasopressor doses to maintain sufficient blood pressure without causing excessive vasoconstriction that could worsen ischemia. The system's ability to identify ischemic areas through oxygen sensors allows for precise adjustment of vasopressor therapy to minimize the risk of further tissue or organ damage, ensuring that perfusion is adequately maintained where it is most needed.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It will be appreciated that the exemplary apparatus shown in the drawings are not necessarily drawn to scale, with emphasis instead being placed on illustrating the various aspects and features of the illustrated examples.

FIG. 1 shows an example of a system implanted within a patient's body.

FIG. 2 shows another example of a system implanted within a patient's body.

FIG. 3 shows an example of a device for creating a pressure differential within a patient's body that may be included in a system such as those shown in FIGS. 1 and 2.

FIG. 4 is a table showing exemplary hemodynamic data obtained by creating a pressure differential in the inferior vena cava.

FIGS. 5A and 5B are tables showing exemplary data of the relationship between left atrial pressure and bi-ventricular pacing.

FIG. 6 is a graph showing exemplary computer modeling data relating discordant bi-ventricular function to cardiac output, left atrial pressure, and left atrial plus pulmonary circulation blood volume.

FIG. 7 is a flow chart showing an exemplary method for using pressure measurements, heart rate, and estimates of cardiac output to estimate left-sided filling pressures.

FIGS. 8A and 8B are graphs showing an example of how changes in heart rate and pressure differential affect both the estimated left-sided filling pressure and the cardiac output.

FIG. 9 is a graph showing the relationship between the estimated left sided filling pressures and estimates of cardiac output illustrating an exemplary method for using the slope of this relationship to determine the optimal heart rate and pressure differential.

FIG. 10 is a flow chart showing an exemplary method for determining the optimal heart rate and pressure differential based on measurements and programmable aspects of a system implanted within a patient's body.

FIG. 11 shows another example of a device for creating a pressure differential within a patient's body.

FIG. 12 shows an example of a device creating a pressure differential within the patient body and the mathematical relationship this implies.

FIG. 13 is a chart of data demonstrating the relationship between flow, pressure differential, and radius of a tube.

FIGS. 14A and 14B show an example of an anchoring device opening at a bifurcation and a balloon with embedded sensors.

FIGS. 15A and 15B show an example of a system with three obstruction devices implanted within the inferior vena cava to strategically move blood volume.

FIGS. 16A and 16B are other examples of a system that includes multiple obstruction devices and sensing mechanisms implanted in the body.

FIG. 17 shows an example of the pressure seen by the kidneys and/or renal veins before and after system activation.

FIGS. 18A and 18B are cross-sectional and side views, respectively, of another exemplary system.

FIGS. 19A and 19B show another example of a multi-obstruction device system with or without an anchoring device and with alternative device placement within the body.

FIGS. 20A-20C show an example of an anchoring device with semipermeable hollow fiber membranes incorporated as the distal occlusion portion. Enlargement of this portion occludes the superior aspect of the IVC.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

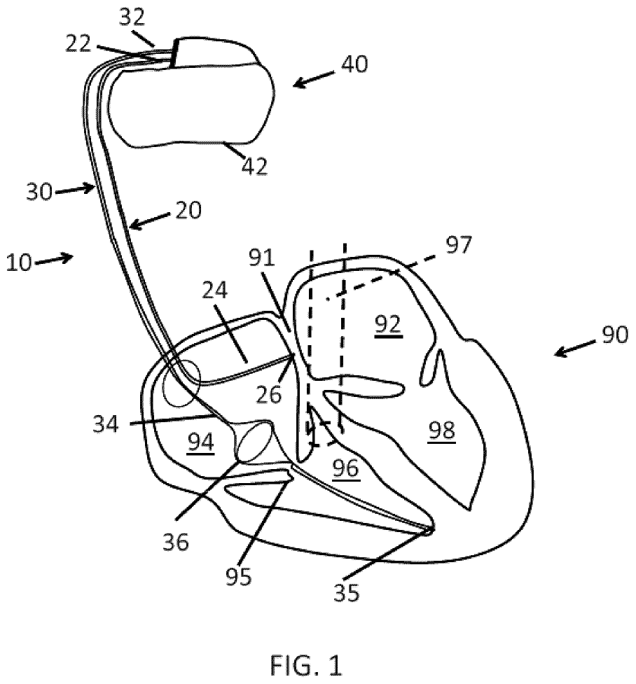

Turning to the drawings, FIG. 1 shows an example of a system 10 that may be implanted within a patient's heart 90 for prevention and/or remediation of heart disease, e.g., for optimizing intra-cardiac filling pressures, heart rate, and/or cardiac output for patients, such as patients suffering from conduction disease, atrial fibrillation, and/or congestive heart failure. In the example shown, the system 10 includes first and second leads 20, 30 coupled a controller 40 for operating the system 10, e.g., to perform the various methods and/or functions described elsewhere herein.

As shown, the first lead 20 includes a first or proximal end 22 coupled to the controller housing 42 and a second or distal end 24 sized for introduction into the patient's heart 90, e.g., into the right atrium 94. The distal end 24 may include one or more features, e.g., a screw tip or other anchor (not shown), for securing the distal end 24 relative to the heart 90, e.g., into the septum 91 between the right atrium 94 and the left atrium 92. In addition, one or more sensors 26 (one shown) may be provided on the distal end 24 that may be coupled to the septum 91, e.g., to provide signals corresponding to the pressure within the left atrium 92. One or more wires or other conductors (not shown) may extend between the proximal and distal ends 22, 24 to communicate the signals from the sensor(s) 26 to the controller 40.

Similarly, the second lead 30 may include a proximal or first end 32 coupled to the housing 42 and a distal or second end 34 sized for introduction into the patient's heart 90, e.g., into the right atrium 94, through the tricuspid valve 95 into the right ventricle 96. The second lead 30 includes an expandable member 36 on the distal end 34, e.g., offset proximally by a predetermined distance from a distal tip 35 of the second lead 30 such that the expandable member 36 is located within the right atrium 94 and/or the tricuspid valve 95. As described further below, the expandable member 36 may be sized to expand and at least partially fill a chamber or other body lumen within the patient's body, e.g., within the right atrium 94. Optionally, similar to the first lead 20, the second lead 30 may include one or more features, e.g., a screw tip or other anchor (not shown), on the distal tip 35 to secure the distal end 34 within and/or relative to the patient's heart 90, e.g., the wall of the heart 90 within the right ventricle 96, similar to pacing leads.

Alternatively, the first and second leads may be provided on a single device with a branched distal end (not shown), e.g., with the one or more sensors on a first branch and the expandable member on a second branch of the lead, as described elsewhere herein.

In one example, the expandable member 36 may be a compliant balloon configured to expand between a collapsed configuration and one or more expanded configurations, e.g., that at least partially fill the right atrium 94 (or other body lumen) and/or occlude flow into or through a body lumen within or adjacent the heart 90, as described further elsewhere herein. For example, the expandable member 36 may be a balloon formed from an elastic material such that the balloon 36 may be inflated to a variety of different expanded sizes, e.g., to change the volume the balloon 36 occupies within the right atrium 94 and/or enhance sealing engagement between the balloon 36 and surrounding tissue. Alternatively, the expandable member 36 may be a non-compliant balloon formed from substantially inelastic material such that the balloon 36 expands to an expanded configuration having a pre-configured shape and/or size. Alternatively, the expandable member 36 may be inflated or deflated based on measured data and hematological parameters including but not limited to venous pressure, blood pressure, cardiac output, heart rate, and oxygen saturation.

In this example, the second lead 30 may be a catheter including an inflation lumen (not shown) extending between the proximal end 32 and the distal end 34 and communicating with an interior of the balloon 36. The controller 40 may include a pump or other source of inflation media (not shown) therein, which may be delivered into and/or removed from the balloon 36 via the inflation lumen, e.g., to direct the balloon 36 between the collapsed configuration and the one or more expanded configurations.

Alternatively, the expandable member 36 may be a mechanically expandable device, which may expand and/or otherwise change shape in response to the controller 40. For example, one or more cables or other actuator members (not shown) may extend between the proximal and distal ends 32, 34 of the second lead 30, e.g., coupled to an actuator (not shown) within the housing 42 and an expandable frame (e.g., covered by a membrane, not shown) or other structure of the expandable member 36. In this manner, the controller 40 may actuate the expandable member 36 to expand and collapse the expandable member 36 between one or more different sizes and/or shapes. The expandable member 36 may be a temporary or a permanently implantable device.

Optionally, the controller 40 may be part of a pacing system, which may include one or more pacing electrodes (not shown) on one or both of the leads 20, 30, and/or other leads (not shown) implanted within the patient's heart 90. Examples of such systems and components that may be incorporated into the systems described herein are disclosed in U.S. Pat. Nos. 4,467,807, 4,535,774, 5,447,524, 8,043,360, and 8,406,879, and U.S. Publication Nos. 2003/7159593, 2004/0111006, 2005/0049692, 2006/0206029, 2010/0056999, 2010/0057192, 2011/0190874, and 2012/0165928, the entire disclosures of which are expressly incorporated by reference herein. For example, a plurality of electrodes (not shown) may be provided on the first and/or second leads 20, 30, as desired, e.g., within the right atrium 94, the right ventricle 96, and/or elsewhere to provide pacing signals to the heart 90. In an exemplary mode of operation, the controller 40 may acquire signals from the pressure sensor(s) 26. In one example, the sensor(s) 26 may acquire electrical or other signals via the septum 91 that correspond to pressure within the left atrium 92 or left ventricle 98. Alternatively, a pressure sensor on the distal end 24 may be directed through the septum 91 into the left atrium 92 (not shown) to acquire pressure data directly.

Based at least partially on the signals acquired by the sensor(s) 26, the controller 40 may expand the expandable member 36 to one or more desired sizes. For example, if the expandable member 36 is a balloon positioned within the right atrium 94, the controller 40 may gradually increase and/or otherwise modify the size of the balloon 36 to at least partially fill the right atrium 94. As the balloon 36 fills the right atrium 94, the volume may be adjusted to reduce or otherwise provided a desired pressure gradient across the expandable member 32. Alternatively, the balloon 36 may be expanded within the tricuspid valve 95 to induce a pressure gradient across the valve 95, i.e., from the right atrium 94 into the right ventricle 96.

The resulting pressure gradient resulting from the expandable member 36 may cause blood volume to translocate from the pulmonary artery 97, lung circulation (not shown), left atrium 92, and/or left ventricle 98 to the venous system behind the expandable member 36. In this manner, the controller 40 may adjust the size and/or configuration of the expandable member 36 over time, e.g., based upon cardiac output trends and/or pressure measurements within the left atrium 92 or left ventricle 94 and, optionally based on activity level or other aspects of the patient's condition. However, such adjustments may be independent of heart beat, i.e., the expandable member 36 may not expand and collapse in synchronization with beating of the heart 90, but instead may be maintained at a desired size, which then be adjusted slowly over time based on pressure changes. For example, the controller 40 substantially continuously or periodically or otherwise intermittently acquire pressure data and adjust the size of the expandable member 36 at a rate substantially slower than the patient's heart rate, e.g., over multiple heart beats, seconds, or minutes.

Furthermore, in patients with volume overload, providing more pre-load to the right ventricle 96 may cause an increase in right ventricular afterload with no change in cardiac output. This may result because the left ventricle 98 is on the “flat” part of the Frank-Starling curve. Providing more pre-load to the right ventricle 96 therefore results in higher intracardiac filling pressures but no change in cardiac output. Unlike conventional treatments, when the expandable member 36 decreases the pressure gradient and thereby increases the filling pressure to the right ventricle 96 without a change in cardiac output (for example by measuring electrical impedance change from electrodes 82), these measurements may be used to identify the heart 90 is not benefitting from additional filling pressure. In addition, the system 10 may monitor changes in pulmonary or left-sided filling pressures in response to small changes in right ventricular filling pressures, thereby again identifying when the heart 90 is not benefitting from the additional filling pressure without measuring changes in cardiac output. In these circumstances, the controller 40 may be programmed to change the expandable member 36 to re-increase the pressure gradient, e.g., in order to collect more extraneous blood volume to the high capacitance venous system below the expandable member 36. In these examples and others, the system 10 may identify the optimal filling pressure and/or heart rate in order to optimize the cardiac output without causing significant elevations in left-sided filling pressures.

Optionally, the controller 40 may include one or more communication devices, e.g., a transmitter and/or receiver, for communicating with external devices (not shown). For example, the controller 40 may transmit one or more of pressure, heart rate, and cardiac output data to an external recording device, which may be used to monitor the patient over time. In addition or alternatively, the controller 40 may be sent instructions from an external device, e.g., to change parameters related to operation of the expandable member 36, pacing parameters, and the like.

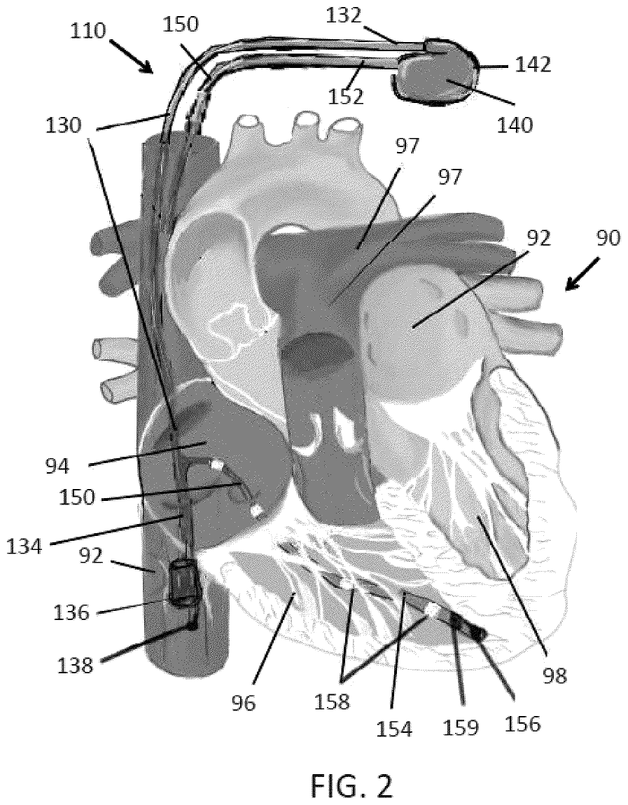

Turning to FIG. 2, another example of a system 110 is shown that may be implanted within a patient's body, e.g., to determine the heart rate and/or the pressure gradient based at least in part on the measured pressures or impedance measurements. Generally, the system 110 includes one or more leads, e.g., a first or flow impedance lead 130 and a second or pacing/sensing lead 150, and a controller 140, similar to other examples herein.

The controller 140 generally includes one or more processors, memory, and/or other components (not shown) sealed within a housing 142, which may be sized to be implanted within the patient's body, e.g., outside but adjacent to the heart 90. The housing 142 may include one or more additional components for operating the system 110, such as a battery or other power source, one or more transmitters and/or receivers, and the like (all not shown). Optionally, an accelerometer may be provided, e.g., also within the housing 142 and coupled to the controller 140 to measure changes in patient movement, which may be used to estimate patient activity.

The controller 140 is generally coupled to the leads 130, 150 to receive sensory data from the heart 90 and/or deliver therapy to the patient. In one example, the second lead 150 includes a proximal end 152 coupled to the housing 142 and a distal end 154 sized for introduction into the patient's heart 90, e.g., into the right ventricle 96. The second lead 150 includes one or more right ventricular pacing electrodes 156 (one shown) on the distal end 154, which may be coupled to controller 140, e.g., to measure heart electrical activity and/or to pace the right ventricle 96. In addition, the second lead includes a plurality of sensors 158 coupled to the controller 140 to measure electrical impedance, e.g., between pairs of the sensors 158 and/or other pacing components of the system 10, such as the RV pacing electrode 156 and/or the right atrial pacing electrode (not shown). The controller 140 may use changes in the impedance measurements to estimate right ventricular volume, e.g., to determine stroke volume. The cardiac output of the right ventricle 96 approximates the cardiac output of the left ventricle 98, and, therefore, the total cardiac output may be estimated by measuring the impedance changes along the sensors 158. Optionally, the controller 140 may also be connected to a pacing lead (not shown), e.g., positioned within the right atrium (94) to sense and pace the heart atria.

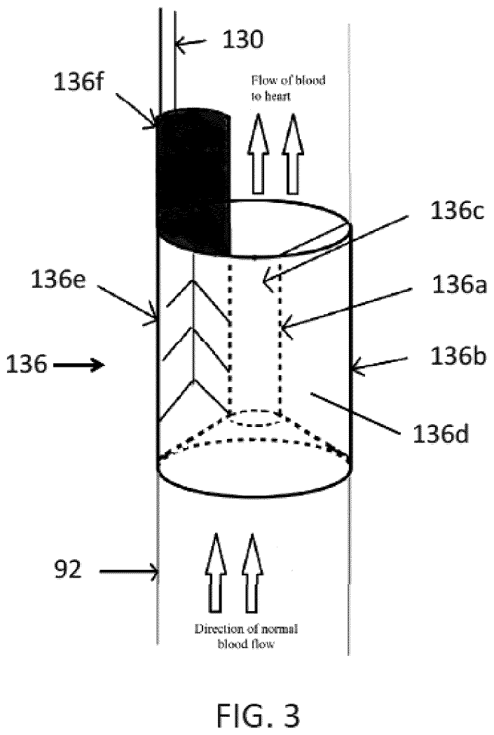

The first lead 130 may include a proximal end 132 coupled to the housing 142 and a distal end 134 sized for introduction into a body lumen, similar to other examples herein. The distal end includes a flow impedance device 136 that may be controlled by the controller 40 to induce a pressure gradient to blood flow through the device 136, such as located in the inferior vena cava 93. The flow impedance device 136 may be configured to control the pressure gradient in a variety of methods, as described further elsewhere, e.g., with reference to FIG. 3. Configuration changes in the flow impedance device 136 may translocate more total body blood volume to the high capacitance located behind the flow impedance device 136.

The first lead 130 may also include a sensor 138 on the distal end 134 distally beyond the flow impedance device 136. The sensor 138 may be coupled to the controller 140 to measure the pressure of blood beyond the flow impedance device 136, e.g., upstream within the inferior vena cava 92.

Similarly, the right ventricular lead 150 may also include a sensor 159 on the distal end 154 coupled to the controller 140 and configured to measure the pressure within the right ventricle 96. In one example, the sensor 159 may be configured to measure pressure waveforms within the right ventricle 96 and the controller 140 may use waveform analysis to estimate the pressure waveforms in the pulmonary artery 97. Utilizing estimates of cardiac output and pulmonary artery pressure waveforms may be used to estimate the pressure of the left atrium 92 and left ventricle 98 e.g., as described elsewhere herein.

Using the estimates of intracardiac filling pressures from the pressure recordings determined from the sensor 159 and estimates of the cardiac output from measurements along the sensors 156, the controller 140 may adjust the pressure differential from the flow impedance device 136 and/or pacing from the pacing electrode 156 with the resulting changes in cardiac output and filling pressure monitored and recorded. For example, from these measurements, the optimal pressure differential, heart rate, and/or cardiac output may be determined and implemented.

In another example, when the pacing lead 150 is introduced to pace the right ventricle 96, the pacing electrode 156 may be positioned along the base, the septum, or the outflow tract of the right ventricle 96. Since the balance of ventricular function is more important than the absolute pumping function, pacing the heart 90 at different locations may worsen right ventricular stroke volume. Therefore, in circumstances where the right ventricular function exceeds the left ventricular function, pacing of the right ventricle 96 at different locations may cause irregular activation of the right ventricle 96 in order to equalize the function of each ventricle. The method may lower left-sided filling pressures.

Turning to FIG. 3, an example of a flow impedance or pressure differential device 136 is shown that may be provided on the first lead 130. As shown, the flow impedance device 136 generally includes an inner membrane 136a and an outer membrane 136b, which are sized and/or otherwise configured to be placed within the inferior vena cava 93, e.g., using a percutaneous method. The inner and outer membranes 136a, 136b may be concentrically disposed relative to one another such that the inner membrane 136a defines an inner flow passage 136c through the flow impedance device 136 and a region 136d is defined between the inner and outer membranes 136a, 136b, e.g., having an annular or other shape.

The flow impedance device 136 also includes a pressure control mechanism 136e, e.g., disposed within the region 136d and an electrical motor or other actuator 136f coupled to the pressure control mechanism 136e. For example, the actuator 136f may be coupled to the controller 140 (shown in FIG. 2) for operating the pressure control mechanism 136d within the flow impedance device 136, e.g., by converting electrical energy from the controller 140 to cause a mechanical change in the pressure control mechanism 136e.

In examples, the pressure control mechanism 136e may be configured for changing one or more of the cross-sectional area to the inner flow passage 136c, changing the direction or length of blood flow, requiring pressure from below the flow impedance device 136 to move or change an aspect of the pressure control mechanism 136e, and/or changing the orientation of the pressure control mechanism 136e. For example, the pressure control mechanism 136e may include a set of struts or other structures that mechanically expand or contract to cause the inner membrane 136a to move inwardly or outwardly relative to the inner flow passage 136c, thereby constricting or opening the inner flow passage 136c. Using one or more of these mechanisms, the flow impedance device 136 may induce a pressure gradient such that the mean pressure across the flow impedance device 136 may be controlled. Because the capacitance of the venous system connected to the inferior vena cava 93 below or upstream of the flow impedance device 136 is much higher than the arterial system, small changes in the pressure gradient result in a large translocation of blood volume from the pulmonary and arterial system to the venous system below or upstream of the flow impedance device 136 and/or inferior vena cava 93. Thus, the operation of the flow impedance device 136 may result in an effective “mechanical diuresis.”

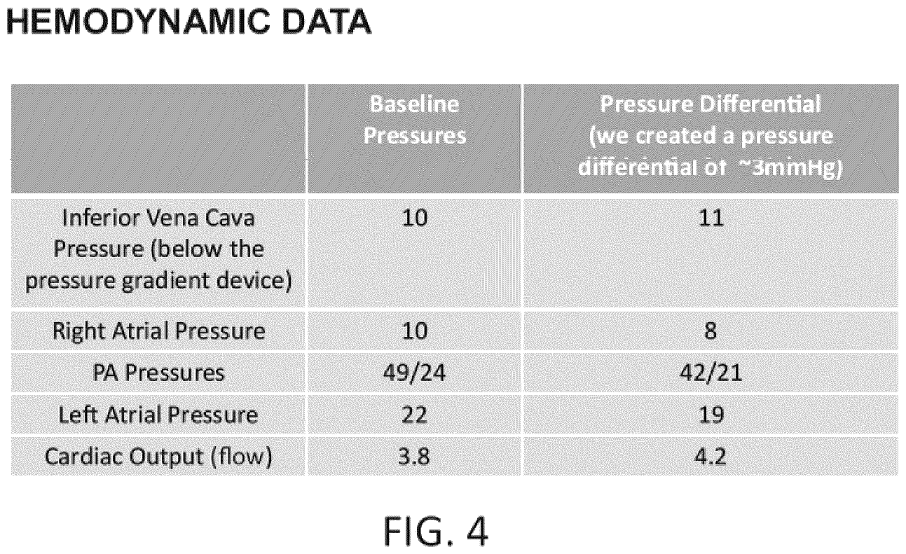

FIG. 4 shows exemplary pressure recordings obtained during experiments in which a pressure differential was created along a device placed in the inferior vena cava 93, similar to the flow impedance device 136 shown in FIG. 3. The pressure differential imposed across the device was approximately 3 mmHg. The subsequent intracardiac filling pressures revealed a reduction in right atrial pressure, a reduction in pulmonary artery pressures, and a reduction of left atrial pressure. Thus, with the pressure differential, a higher cardiac output was created and a higher flow resulted through the flow impedance device.

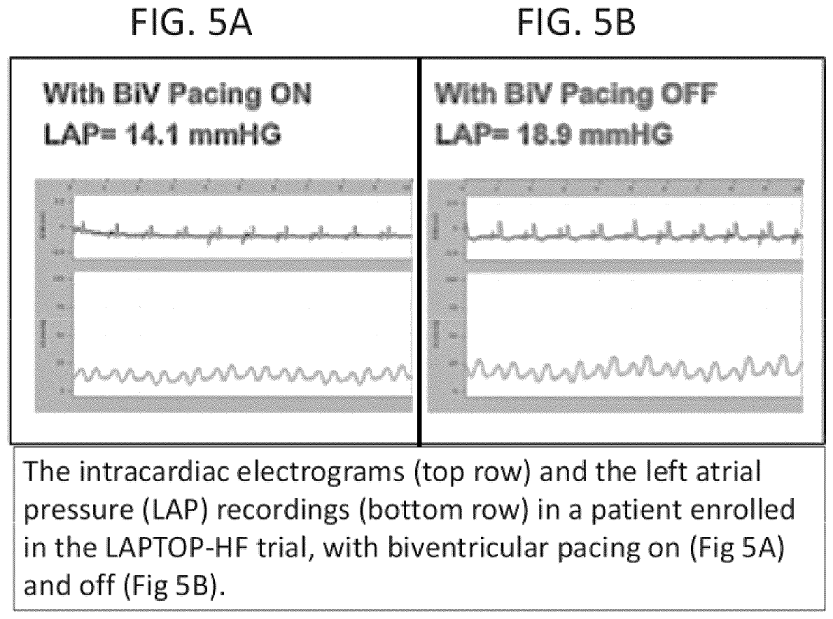

FIGS. 5A and 5B are tables showing published data relating left atrial pressure to bi-ventricular pacing. As can be seen, bi-ventricular pacing is associated with a reduction in left-sided filling pressures. This is likely the primary method by which bi-ventricular pacing improves symptoms and exercise capacity. An interactive device that can maintain intra-cardiac pressures may achieve similar benefits of bi-ventricular pacing. Therefore, patients who are not candidates for bi-ventricular pacing may benefit from an interactive device that creates a pressure differential to blood flow.

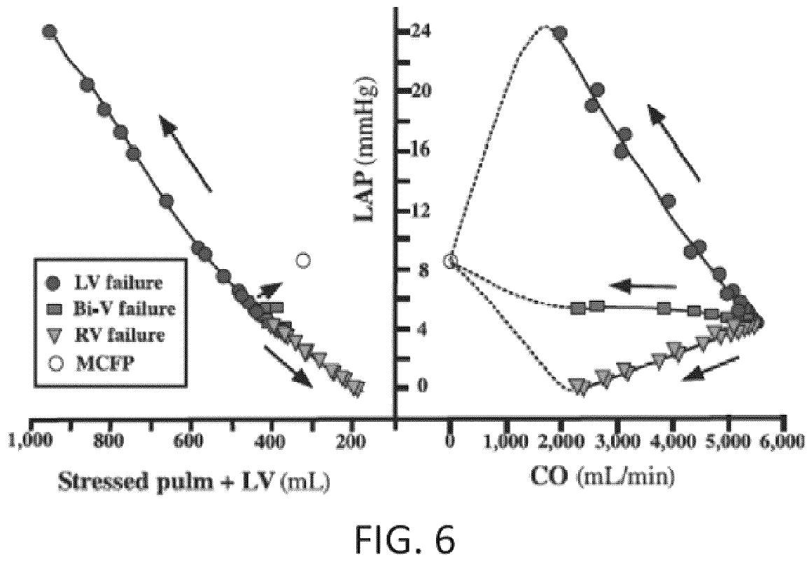

FIG. 6 is a graph showing computer modeling data relating discordant bi-ventricular function to cardiac output, left atrial pressure, and left atrial plus pulmonary circulation blood volume. As can be appreciated, the relationship between ventricular function is more important in determining intra-cardiac filling pressures compared to absolute ventricular function. Therefore, a device that monitors the function of the heart and intracardiac filling pressures may be able to alter the heart rate and intracardiac filling pressures in order to help to balance ventricular function to improve optimize cardiac output and intracardiac filling pressures.

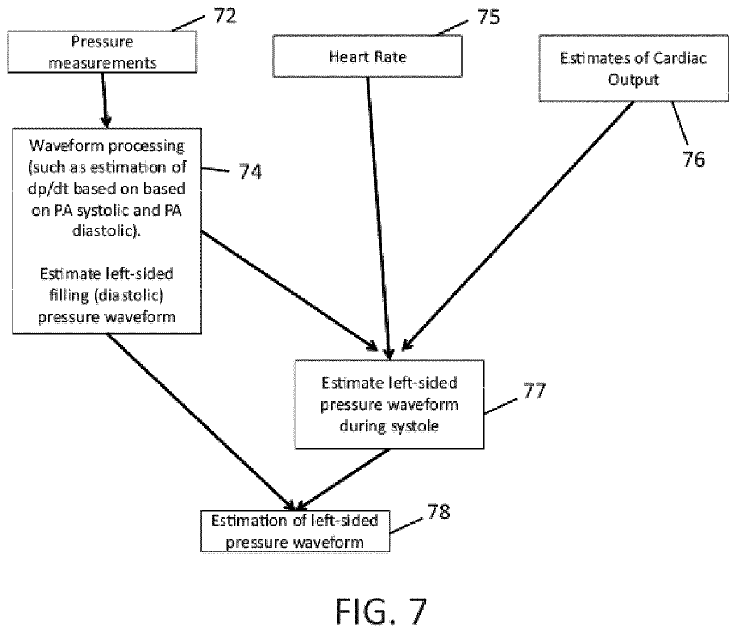

Turning to FIG. 7, an exemplary method is shown in which pressure measurements from the right ventricle, obtained at step 72, e.g., using any of the systems described herein, may be combined with cardiac output measurements in order to estimate the left-sided pressure waveform. At step 74, the pressure waveform from the right ventricle may be used to estimate the pulmonary artery (PA) diastolic pressure (which is the pressure when the right ventricular volume begins to decrease) as well as the pulmonary artery (PA) systolic pressure (which is the peak pressure measured in the right ventricle).

At steps 75 and 76, the heart rate and cardiac output (again obtained using any of the systems herein) may then be used to estimate the rate of pressure decline (dp/dt), which may be used to estimate left-sided diastolic pressure waveforms, at step 77. Furthermore, by combining systolic waveforms with estimates of stroke volume, left-sided systolic waveforms may be estimated at step 78. Therefore, the maximum left-sided filling pressures and effective left-sided filling pressures (LVEDP) may be estimated using any of the systems described herein.

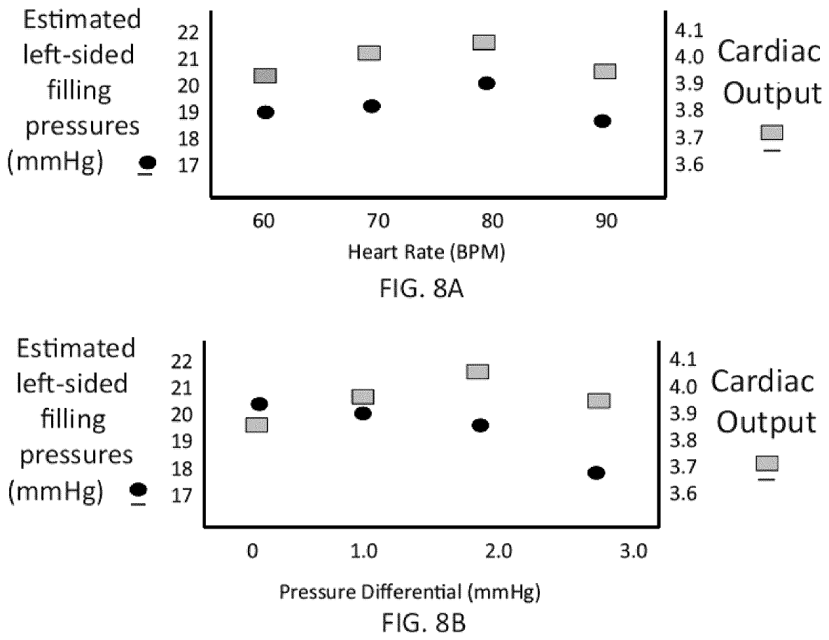

FIGS. 8A and 8B are graphs that show how slight changes in heart rate and the pressure differential may cause changes in both the estimate of left-sided filling pressures (in mmHg) and the cardiac output (in liters a minute). Both the heart rate and the pressure differential may be gradually changed over time, e.g., using the systems and methods described herein. With any change, a few minutes may be required in order for the body to create a new equilibrium, at which time the measurements may be recorded again. The combinations of heart rate and pressure differential may be gradually altered in order to create a series of relationships between the estimated left-sided filling pressures and cardiac output. This curve may be determined for various levels of patient activity. Once the optimal left-sided filling pressures to cardiac output is determined at the determined level of patient activity and total body blood volume, the required heart rate and pressure differential may be maintained.

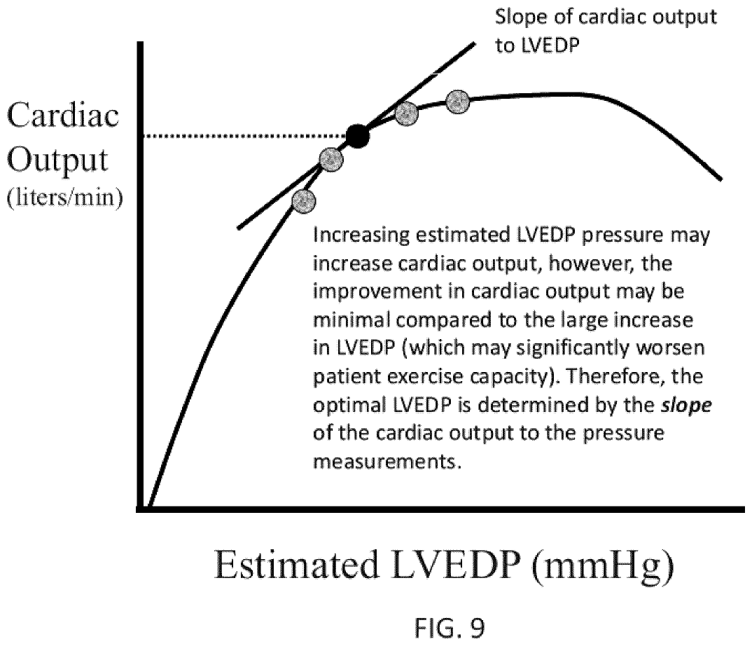

FIG. 9 is a graph that illustrates an exemplary relationship between the measurements obtained in FIG. 5 at one level of patient activity and total body blood volume. As shown in FIG. 6, the estimated cardiac output may be determined for various estimates of the left-sided filling pressure and the left ventricular end diastolic pressure (LVEDP). The resulting curve shown in FIG. 9 is, in essence, the Frank-Starling curve for this patient's level of activity and volume status. Note that at lower pressures, an increase in LVEDP results in an increase in cardiac output. However, at some point, increasing LVEDP results in mild improvement in cardiac output. At high filling pressures, increasing the LVEDP even more may actually decrease cardiac output. Increasing the cardiac output may improve oxygen delivery to tissue and improve kidney function, which may help with diuresis in heart failure patients. However, the LVEDP that optimizes cardiac output may be significantly high to worsen respiratory status. Therefore, the slope relating the LVEDP to the cardiac output may be used to estimate the optimal LVEDP and cardiac output. The slope of the pressure/output curve may be adjusted and determined empirically by a controller of the system at various levels of patient exercise.

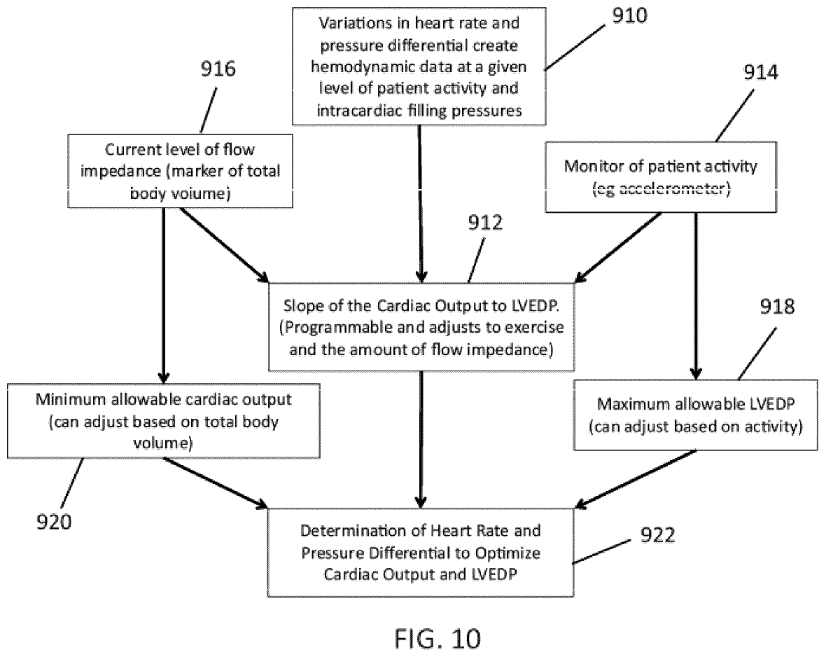

Turning to FIG. 10, an exemplary method is shown for determining optimal heart rate and/or pressure differential, e.g., using any of the systems described elsewhere herein. The controller of the system may operate the flow impedance device and other components to enable the desired data to be acquired and calculations to be performed.

Variations in heart rate and pressure differential are used in order to estimate the relationship between left-sided filling pressures and cardiac output. For example, at step 910, heart and pressure differentials obtained using the system, e.g., across the flow impedance device 136 shown in FIG. 3, may generate hemodynamic data at a given level of patient activity and intracardiac filling pressures. At step 912, the controller may then use the slope of this relationship (e.g., as demonstrated in FIG. 9) to determine the optimal left-sided filling pressure and cardiac output.

Optionally, at step 914, an accelerometer implanted in the patient's body, e.g., within the housing 142 of the controller 140 shown in FIG. 2, may be used to identify the patient's level of activity. For example, the controller 140 may acquire data from the accelerometer indicating that the patient is resting (e.g., no substantially signals from the accelerometer) or active (or the controller 140 may determine various levels of activity based on the signals from the accelerometer). In addition, at step 916, the controller 140 may identify the current level of flow impedance from the flow impedance device 136, which may provide an estimate of the total body volume of the patient.

At step 918, the controller 140 may also determine the maximum allowable left-sided filling pressure and minimum cardiac output allowable. These values may also be dependent on the level of patient activity and the total body blood volume. That is, when the patient is at an increased level of activity, the controller may accept a higher maximum of left-sided filling pressure. At step 920, when the patient has an increased level of total body volume (as evidenced by a high pressure differential), the minimum cardiac output may be raised in order to encourage diuresis. At step 922, these values are then incorporated into the algorithm generated by the controller and used to determine the optimal heart rate and pressure differential, e.g., to optimize cardiac output and/or left ventricular end diastolic pressure (LVEDP).

Once calibrated, pressure differential and heart rate may be anticipated based on patient activity and intracardiac pressures in order to optimize filling pressures and/or cardiac output. Over time, the decreased left atrium and left ventricle filling pressures may lead to beneficial myocardial remodeling. This may improve myocardial function and/or decrease arrhythmias and/or ectopic beats.

As a result of the systems and methods herein, diastolic and systolic function may improve over time. Furthermore, device pacing and pressure control algorithms may be developed to accelerate this process (similar to how exercise is known to improve diastolic dysfunction).

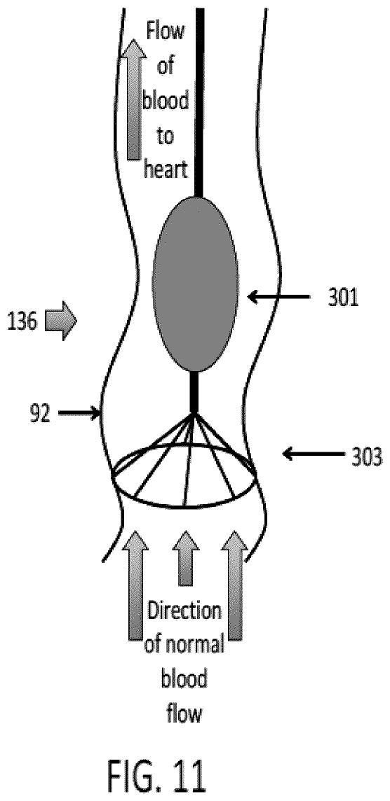

Turning to FIG. 11, another example of a flow impedance or pressure differential device 136 is shown. In this example, the flow impedance device 136 includes a balloon component 301 anchored by an anchoring system 303 within the inferior vena cava 93. The balloon component 301 may be inflated by gas and/or fluids. The anchoring component 303 may be formed from various materials, such as Nitinol or other elastic material. The anchoring system 303 enables a balloon component 301 to control blood flow or pressure without dislodging or bucking to the direction of blood flow. This system 136 may include various sensors and/or fluid filled lumens in order to transmit physiological or device information back to the processor.

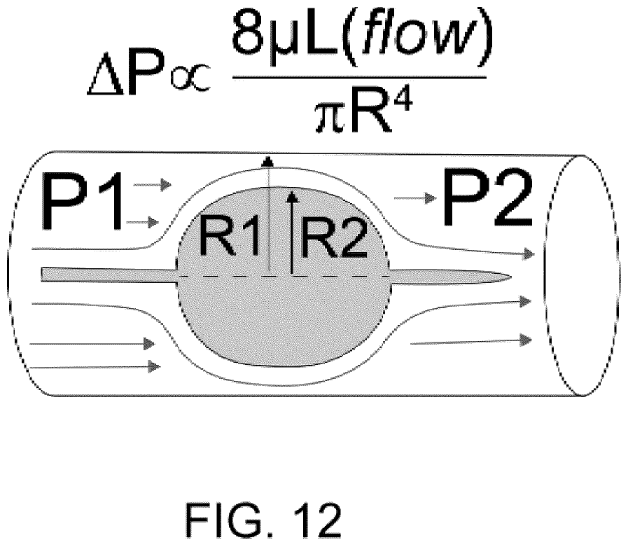

FIG. 12 shows an example of the relationship between pressure, flow, and balloon radius, which may be the basis for calculating cardiac output from inflating the balloon and measuring pressures. The equation is based on Poiuselle's Law, demonstrating there is a proportional relationship between changing pressure differences and flow (cardiac output) and radius (which varies as the balloon or inflation device changes size).

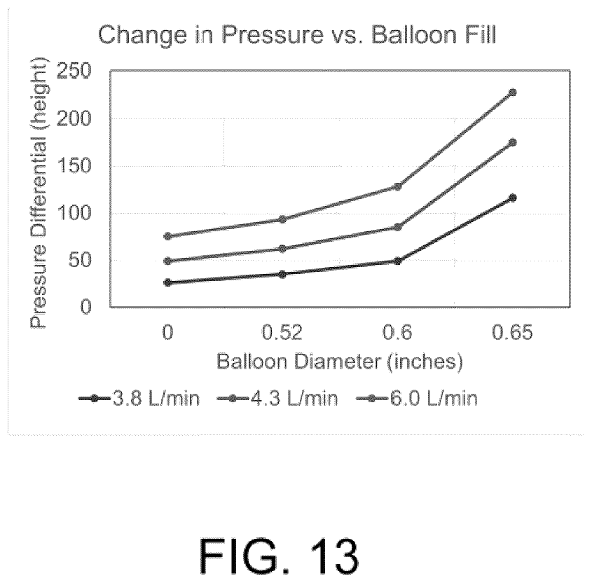

FIG. 13 demonstrates exemplary data of the relationship that can be recorded when perturbing the inflation device and recording pressure differential and the relationship to flow of the fluid through a tube. This relationship may be the basis on which the device is expanded or deflated based on hematological parameters.

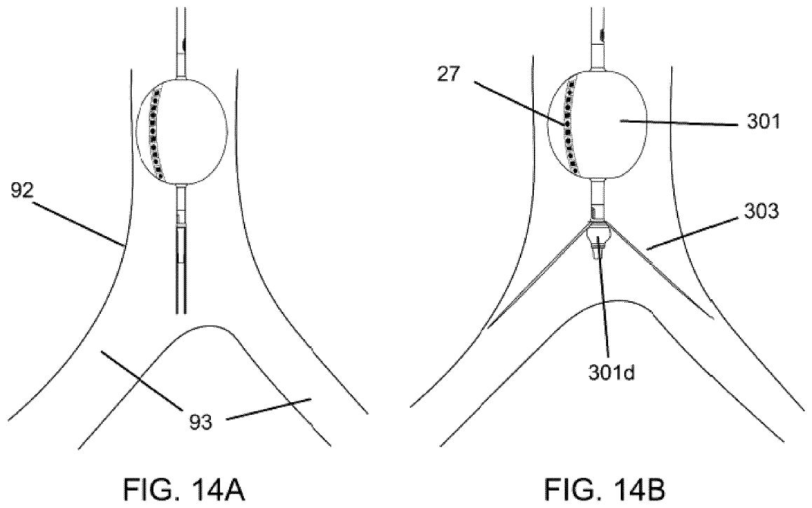

Turning to FIG. 14, an obstruction device 301 is shown (such as an inflatable balloon) on a catheter inserted into the IVC 92 with an anchoring system 303 which can be deployed at a bifurcation such as the common iliac veins 93. In this example, device 301 has a plurality of sensors 27 embedded on or encased in or within the surface. Not pictured, the sensors may also be encased or embedded within or on the catheter. In one or more examples, sensors 27 may include ultrasonic, pressure, optical, magnetic, impedance, resistance, piezoelectric, or other types of sensing. The sensors may be used to detect blood flow rate, blood pressure, or blood contents. In particular, optical sensing has been used for the detection of oxygen saturation, pH and lactate levels, hemoglobin concentration, glucose, coagulation, lipids, metabolites, and other concentrations within the blood, which could be used for monitoring and diagnosis of patients while the system is deployed.

In FIG. 14A, device 303 is not yet deployed to anchor in the bifurcation. In FIG. 14B, a small balloon 301d is expanded to deploy the anchoring mechanism 303. Not pictured, the anchoring mechanism in some examples may be one or more of a variety of shapes and/or materials. Additionally or alternatively, the device may be self-expanding, expanded by a balloon or other mechanism, pushed or pulled from the proximal end to deploy, or screwed in place. Anchoring device 303 may include wires or tethers that engage with the walls of the vessels, or they may screw into the vessel wall, with a helical or screw-like shape. In some examples, it is an expanding stent or filter-like device, whether self-expanding or expanded by a balloon, push/pull, screwing, or other actuation.

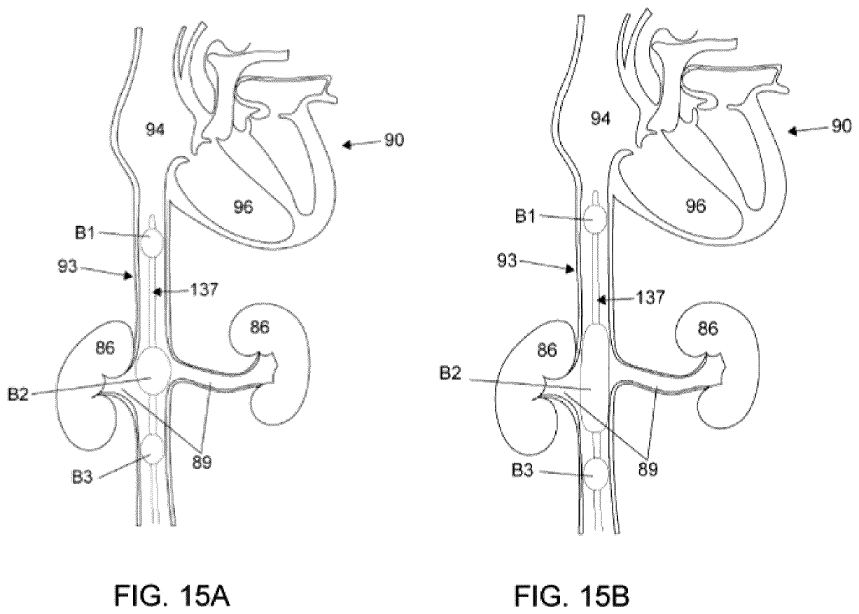

Turning to FIG. 15A, a three-balloon system 137 is shown deployed in the IVC 93 in which one balloon B1 is deployed distal to the renal veins 89, one balloon B2 is located at the renal veins 89, and one balloon B3 is proximal to the renal veins 89. In FIG. 15B, the balloon B2 located at the renal veins has an elongated shape to encompass more of the IVC 93 around the renal veins 89. In some examples, B1, B2, and B3 are expandable members that can be mechanically, electrically, pneumatically, and/or magnetically actuated to fully or partially occlude the vena cava. Distal expandable member, or balloon, B1 may be used to measure central venous pressure from the right atrium 94 in the heart 90.

In some examples, B1 is used to optimize CVP to a set value or based on the patient's Frank-Starling curve. In certain examples, the system 137 has various “modes” that will automatically or manually enable specific functions, such as protecting the kidneys 86 by inflating the expandable members in such a way to reduce volume, and/or protecting the lungs by reducing CVP. Some patients, such as those who have been intubated, may require higher CVP to preserve cardiac function. In this and other examples, anticoagulation may be used when balloons are inactive to prevent clotting. Alternatively or in addition, the expandable member or balloon may be pulled taut by internal or external mechanisms to anticoagulated. In some examples, system 137 may include multiple telescoping catheters, each with at least one balloon or expandable member that is pulled into the current outermost catheter or next outer catheter when the expandable member is not active.

In some examples, the system may include multiple balloons placed within the inferior vena cava to create controlled pressure gradients and facilitate blood flow. The system includes a pumping balloon positioned between two occlusion balloons. The lower balloon is placed below the renal veins to create a partial or complete occlusion of the IVC, while the upper balloon is positioned above the pumping balloon to provide a downstream obstruction. This configuration is useful to maintain an area of low pressure at the level of the renal veins, promoting venous return from the kidneys to the heart. The synchronized inflation and deflation of these balloons generate a peristaltic effect, enhancing blood flow and reducing renal vein pressure. The partial obstructions created by the upper and lower balloons are useful for the effectiveness of the pumping balloon, as they help maintain the necessary negative pressure zone.

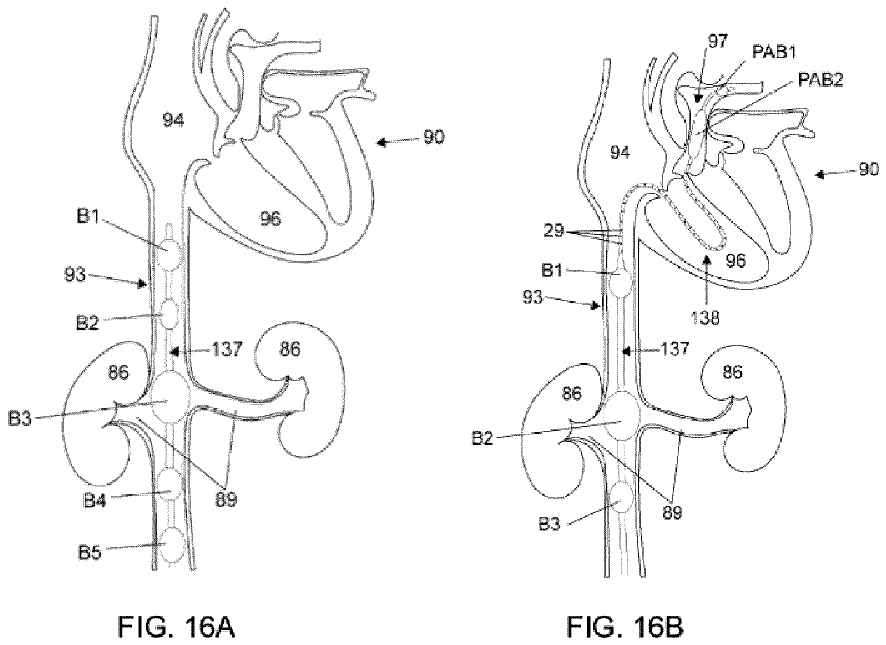

In FIG. 16A, an example is shown with more than three expandable members to create a peristaltic effect. In the example shown, there are five expandable members B1-B5, and in other examples (not pictured), more balloons may line the entire device 137 or be spaced strategically along the elongate member. FIG. 16B demonstrates another example in which a catheter 138 extends from the first device 137 into the pulmonary artery 97. This catheter may have one or more expandable members to either anchor (PAB1) or fully or partially occlude (PAB2) to assist in cardiac function. Device 138 may be lined with impedance sensors 29 or other sensors (oxygen, pressure, etc.) to detect cardiac filling pressures and/or cardiac output. In this and other examples, the device may measure VO2 at all times and correspondingly inflate or deflate balloons based on this and/or other measurements. In these and other examples, balloons may be inflated using gas or liquid, such as saline or helium, from a pumping mechanism located outside the patient's body. The pumping mechanism and/or expandable member actuator will be connected to a controller and monitoring system.

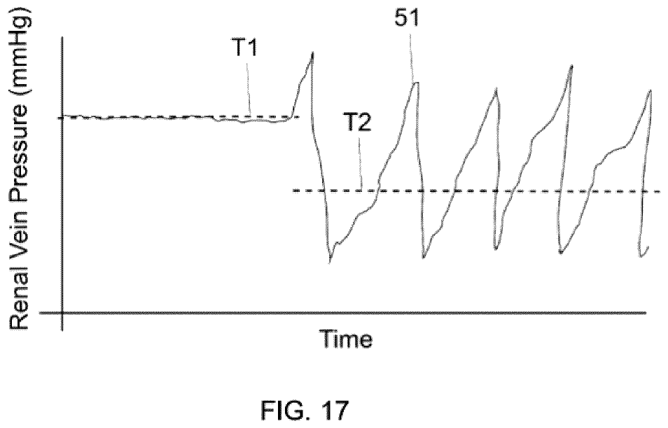

FIG. 17 demonstrates an example of the pressure 51 seen by the kidneys or the renal veins over time. At time T1, before system activation, the kidneys experience elevated pressures which may contribute to acute kidney injury or impaired renal function. At time T2, the average pressure seen by the kidneys may be lower as the balloons in the IVC strategically inflate and deflate to create a negative pressure zone to move blood volume away from the kidneys.

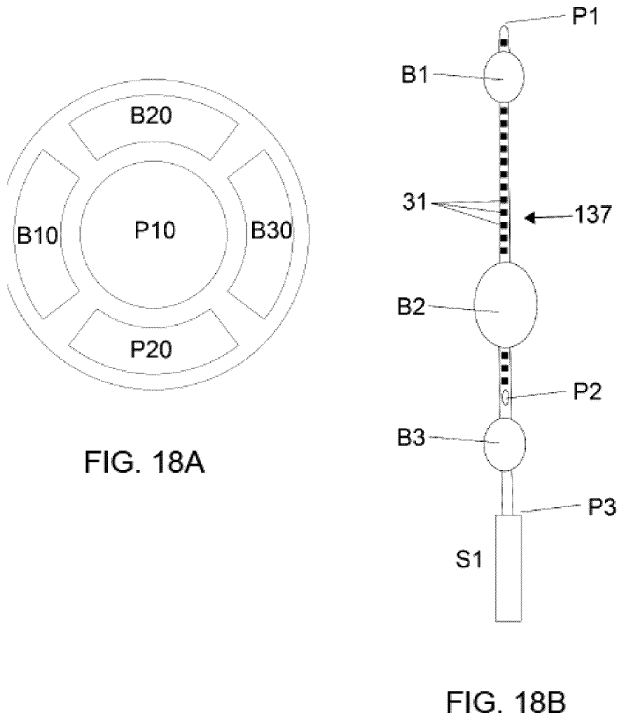

FIG. 18A shows a cross-section of the system 137 shown in FIG. 18B. The cross-section in this example features four lumens corresponding to various locations on device 137. Lumen P10 extends through the length of the device and is open to the distal end P1, which will be used to measure distal pressure with a pressure transducer. Lumen B10 is used to inflate balloon B1, lumen B20 is used to inflate balloon B2, and lumen B30 is used to inflate balloon B3. A port P2 is open on the side of the device 137 proximal to B2 to measure pressure around the renal veins through lumen P20. Proximal pressure P3 is measured from a sheath S1 that is used to introduce device 137 into the IVC.

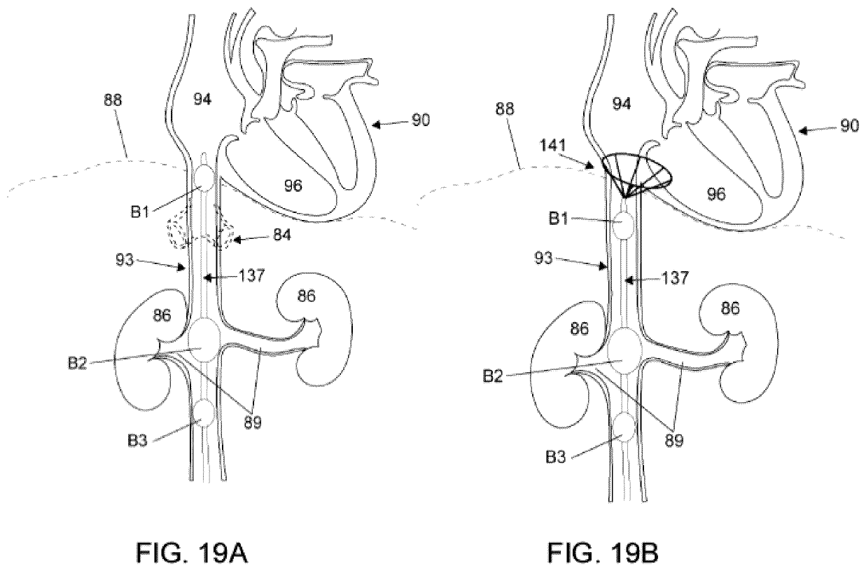

FIG. 19A demonstrates an example the system 137 in which balloon B1 is strategically placed distal to the hepatic veins in order to move excess volume into the splanchnic system. This example may be used independently or in combination with the example shown in FIG. 19B which also shows anchoring.

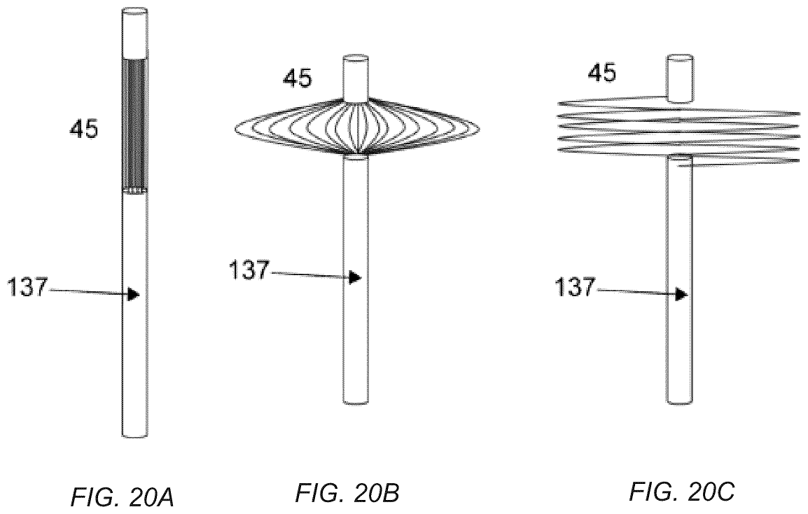

FIGS. 20A-20C demonstrate an example of the system 137 with semipermeable hollow fiber membranes 45. Enlargement of this portion occludes the superior aspect of the IVC. This membrane also enables air flow to remove CO2 and possibly add O2. The balloon pump helps force blood through this occluder to help with gas exchange.

Optionally, any of the systems herein may include at least one additional sensor configured to provide sensor data corresponding to the stroke volume or cardiac output. In one example, the at least one additional sensor comprises a plurality of electrodes configured to measure changes in electrical impedance that correlate with changes in blood volume. The controller may be coupled to the plurality of electrodes and configured to estimate cardiac output by measuring the changes in electrical impedance from the plurality of electrodes. For example, at least one catheter, lead, or other elongate member may have two or more electrodes along the catheter capable of measuring electrical impedance. In another example, the at least one additional sensor may be a flow sensor capable of measuring the blood oxygen saturation, such as with a light oximeter. The controller may be configured to estimate left-sided filling pressures by analyzing sensor data from at least two of these sensors.

In still another example, the at least one pressure sensor may be configured to be located in the pulmonary artery, right atrium, superior vena cava, inferior vena cava, right ventricle, left atrium, and/or coronary sinus (or any vein of the heart), e.g., carried by a lead implanted in the patient's body.

In an example, the at least one adjustable component may be configured to be placed percutaneously and selectively expanded in the vena cava with a mechanism to induce a pressure gradient along the inside of the adjustable component. In one example, the adjustable component may be an expandable member, e.g., an inflatable balloon or a self-expanding mechanism. In another example, the adjustable component may be a tubular member that may be selectively actuated to change shape and/or size. In yet another example, the adjustable component may be connected to an electrical motor that may induce changes to the adjustable component.

In any of these examples, the controller may utilize changes in blood pressure to induce a mechanical change in the adjustable component. For example, filling pressures of the right atrium may fluctuate, e.g., from about 5 mmHg to about 10 mmHg, while the pressure below or upstream of the adjustable device may maintain a pressure of about 10 mmhg. The controller may actuate the flow impedance device to use energy from this higher pressure to create a pressure drop across the device, e.g., to use the pressure differential that occurs during the normal heart cycle to create a pressure gradient.

In yet other examples, the adjustable component may create a pressure gradient by at least one of the following: i. adjusting blood flow impedance across the tricuspid valve; ii. decreasing the size of the right ventricle; iii. adjusting blood flow impedance through the superior vena cava; iv. adjusting blood flow impedance through the inferior vena cava; v. adjusting blood flow impedance through the azygous vein; vi. adjusting blood flow impedance through the right ventricle or tributary; vii. adjusting blood flow impedance through the pulmonary valve and/or pulmonary artery; and viii. adjusting blood flow impedance from the left atrium into a lower pressure conduit.

In addition or alternatively, the blood flow impedance may be adjusted by at least one of: i. inflating a balloon; ii. where the adjustable component has a certain shape, including but not limited to, a doughnut shaped structure where blood flows through the structure; iii. changing the size or orientation of a shunt; iv. shunting the direction of blood flow; v. adjusting the size of a conduit; vi. a rotating propeller; vii. adjusting the cross-sectional area of blood flow; and viii. adjusting a valve, where the valve includes one of a ball valve, shutter valve, butterfly valve, diaphragm valve, gate valve, globe valve, knife valve, spool valve, thermal expansion valve, and a pressure reducing valve.

In other examples, the adjustable component may be located in the patient's inferior vena cava or below the renal veins. Alternatively, the adjustable component may be located outside a blood vessel in the patient's body.

Optionally, in any of these examples, the system may include at least one sensor configured to correlate with patient activity, such as with an accelerometer device.

In accordance with another example, during operation of the system, the controller may change the function of at least one component in order to assess and/or record changes from at least one sensor component. For example, the controller may determine the function of at least one component based on changes in sensor data in response to changes in function of at least one component.

In addition or alternatively, the controller may maintain a minimum acceptable cardiac output and a maximally allowable left-sided filling pressure. For example, the minimum acceptable cardiac output and the maximally allowable left-sided filling pressure may be influenced by sensor data.

In addition or alternatively, the controller may be configured for adjusting the function of at least one component based at least in part on prior recordings from the system and current recordings from at least one sensor.

Optionally, in any of these examples, sensor data and/or device functioning details may be transmitted outside of the patient, e.g., using a transmitter or transceiver carried by the controller or other component of the system. In addition or alternatively, the functionality of the device may be adjusted from outside of the patient's body, e.g., by communicating with the system using a receiver or transceiver carried by the controller or other component of the system.

In another option, in any of these examples, the system may also include one or more pacing electrodes, and the controller may be configured to adjust the pacing parameters of the one or more pacing electrodes in response to sensor data.

In still another example, the adjustable component may be at least partially fabricated from animal tissue (such as bovine or porcine tissue), Nitinol, synthetic fluoropolymer, stainless steel, chromium compounds, and/or various polyesters.

In accordance with another example, a system is provided for implantation in the body of a patient with conduction disease and/or heart failure configured to monitor and/or treat the patient that includes a catheter, lead, or other elongate member sized for implantation within a patient's body, e.g., such that at least one end of the lead is positioned within a chamber of the patient's heart, a blood vessel, and/or other body lumen. The system may include at least one sensor carried by the elongate member and configured to provide sensor data corresponding to pressures within or near the heart. The system also includes at least one adjustable component configured to create a pressure gradient to blood flow carried by the elongate member or a second catheter, lead, or elongate member. The system also includes at least one pacing component configured to at least one of sense and pace the heart, and a controller configured for adjusting the function of at least one component based at least in part on sensor data from at least one sensor.

In one example, the elongate member includes a proximal end coupled to a housing containing the controller and a distal end sized for introduction into the patient's heart, and wherein the at least one pressure sensor and the at least one component are carried by the elongate member. For example, the elongate member may include a first branch on the distal end carrying the at least one pressure sensor, and a second branch on the distal end carrying the at least one component. In one example, the elongate member is a catheter including an inflation lumen extending between the proximal end and the second branch, and wherein the at least one component includes a balloon comprising an interior communicating with the inflation lumen for delivering and removing inflation media into and from the interior.

In another example, the elongate member is a catheter including first and second inflations lumen extending between the proximal end and the second branch, and wherein the at least one component comprises a first balloon comprising a first interior communicating with the first inflation lumen and a second balloon comprising a second interior communicating with the second inflation lumen such that inflation may be delivered into and removed from the first and second interiors to independently expand and collapse the first and second balloons.

Optionally, the system may include an actuator member extending from the elongate member proximal end to the second branch, and wherein the at least one component comprises a mechanically expandable member coupled to the actuator member for selectively expanding and collapsing the expandable member.

In another example, the occlusion mechanism is anchored by an anchoring system. The anchoring system may include a Nitinol like system, e.g., similar to available inferior vena cava filter devices. The anchoring component enables the occlusion device, e.g. an inflatable balloon, to be positioned in the venous system, e.g. the inferior vena cava, and partially occlude blood flow. In this example, the balloon device can be positioned while rotating the device within the venous blood vessel. Therefore, the device can be advanced from jugular veins and/or subclavian vein and positioned within the inferior vena cava without the blood flow moving the device. Furthermore, by having the device inserted from the upper body, this leaves the legs free for ambulation and movement. In other examples, the device can be advanced from placement at the groin through the femoral vein and be fixed in place from the insertion site to prevent it from advancing with the direction of blood flow.

In some examples, the device is temporarily placed as part of a catheter inserted through vein. In other examples, the device is a permanent implant, which remains in the vein and constantly adjusts to the patient hemodynamics. The device, whether permanent or temporary, may also emit drugs to manage patient symptoms.

In accordance with another example, apparatus, system, and methods herein may utilize a variety of methods or devices which can be employed to measure the flow rate of blood in a vessel. These techniques are designed to determine cardiac output directly by measuring the flow rate in a vessel or from within the venous system. The system described may include one or more of these flow rate measurement techniques, or utilize a technique or method not described here. Other types of sensing are described to detect location, perform mapping, and detect blood component levels (such as lactate, pH, and oxygen, among others).

All methods and examples are designed to be minimally invasive, biocompatible, and compatible with standard catheter insertion techniques.

The apparatus, systems, and methods herein may also include ensuring stable placement of a balloon catheter or obstruction device, especially within large vessels like the inferior vena cava (IVC) or at bifurcations such as the common iliac vein, which is crucial for both the efficacy and safety of this system. This will ensure that if the balloon is placed in the vein in such a way that it is opposing blood flow (such as in the inferior vena cava from the IJ), the balloon does not migrate during the procedure. The system may use one or more of these anchoring methods and techniques, or others not listed in this patent.

One example involves two balloons—one for the primary function (e.g., occlusion or drug delivery) and a secondary, smaller balloon to act as an anchor. The secondary balloon is inflated prior to or simultaneously with the primary balloon to provide added stability in a smaller vessel. This example or others may also include expandable anchoring legs, similar to the legs or struts on certain IVC filters, these extendable legs can be incorporated into the catheter's design. Upon reaching the desired location, the legs can be expanded, anchoring the device in place against the vessel wall.

Some examples may include a self-expanding stent or mechanism, wherein a stent-like mechanism made from materials with shape-memory properties (e.g., Nitinol) can be positioned at the catheter tip. Once deployed, this stent expands to fit the vessel, holding the catheter in place. This or other examples may also be deployed using a pushing or pulling device that extends from the catheter. This mechanism would be similar to umbrella mechanisms or certain IVC filter designs. The user can push or pull a component of the device, causing the anchoring mechanism to open or close.

In some examples, there is a helical device or screw for anchoring, in which the tip of the catheter or a device extending from the catheter is designed with helical threads. The catheter is rotated to “screw” into the vessel wall gently, securing it in place. Helical threads may also deploy from the sides of the catheter for anchoring. Other examples include magnetic anchoring, which incorporates magnetic elements either in the catheter or an associated device. External magnets or magnetic implants can be used to hold the catheter in position. Another example uses a temporary adhesive or suctioning mechanism on the tip or section of the catheter or separate anchoring device so that when deployed it can adhere to the vessel wall.

In another example, there are anchoring wires or tethers integrated into the catheter. Once inside the vessel, they can be extended to engage with vessel walls or bifurcations, holding the catheter securely. These tethers may take on a variety of shapes, including but not limited to thin straight wires, circular or elliptical tethers, helically shaped tethers, wide strips, or other geometries. Still another example is friction or texture-based anchoring, where the catheter's external surface or the external surface of an anchoring device has high friction or is textured. The frictional force or texture prevents unwanted movement once the device is positioned. In some examples, the catheter material is stiff enough to withstand the blood flow rate without migration.

These and other examples may also be used to anchor at bifurcations rather than within the vessel being occluded. The catheter can be specially shaped or have expandable side structures to brace against the sides of a bifurcation, such as the common iliac veins from the IVC. As the device or anchoring system is expanded, it makes contact with both branches of the bifurcation, preventing movement. The system may also utilize steering techniques or curved or steerable guidewires for initial placement and/or adjustment during the procedure.

Incorporating oxygen delivery into the catheter can be achieved through several methods: Micro-porous Membrane: The catheter can include a micro-porous membrane that allows oxygen to diffuse directly into the bloodstream. This membrane can be positioned along the length of the catheter that resides within the venous system, ensuring efficient oxygen delivery. Internal Lumens: The catheter can be designed with one or more internal lumens dedicated to oxygen delivery. These lumens can be connected to an external oxygen source, allowing for controlled and continuous oxygen flow through the catheter and into the bloodstream. Oxygen Carriers: The catheter can be coated with or contain oxygen-carrying compounds, such as perfluorocarbons or hemoglobin-based oxygen carriers, which can release oxygen directly into the blood upon contact. Oxygen Diffusion: The catheter can include sections made of materials that facilitate the diffusion of oxygen from an internal lumen to the external environment (i.e., the blood). This design can ensure that oxygen is gradually and efficiently transferred to the blood as it flows past the catheter. Integrated Sensors: In addition to delivering oxygen, the catheter can be equipped with integrated sensors to monitor blood oxygen levels in real-time. These sensors can provide feedback to regulate the oxygen delivery rate, ensuring optimal oxygenation without over-delivery, which can be particularly beneficial for maintaining precise oxygen levels during medical procedures.

Incorporating oxygen delivery mechanisms into the catheter can enhance the therapeutic capabilities of the device, providing critical oxygenation support while simultaneously monitoring and managing hemodynamic parameters. This integrated approach can be especially advantageous in managing patients with complex cardiovascular and respiratory conditions, improving overall patient outcomes.

In some examples, the device also includes targeted drug delivery capabilities. In conjunction with or as an alternative to systemic diuretics, it can deliver medication directly to the kidneys or other target organs, promoting localized diuresis or enhancing cardiac function while minimizing systemic side effects. This targeted approach allows for personalized treatment plans that can be optimized based on real-time physiological data.

Some examples may further expand treatment by integrating remote patient monitoring and providing educational resources. Patients can transmit physiological data to their healthcare providers, enabling remote monitoring and timely adjustments to therapy. Educational resources empower patients to actively participate in their care, improving self-management skills and overall well-being.

In some examples, the systems and methods may include a multi-balloon (or other occlusion device) catheter system designed to create controlled pressure gradients within the inferior vena cava (IVC). The system includes a proximal balloon positioned below the renal veins to completely or partially occlude the IVC, a middle balloon at the level of the renal veins to function as a pumping mechanism, and a distal balloon positioned higher up in the IVC to maintain a negative pressure environment and measure cardiac output. The proximal balloon can remain inflated for extended periods (e.g., 5-10 minutes or longer) to evaluate the impact on venous pressure and renal function.

By strategically inflating and deflating the balloons in a coordinated sequence, the device creates a peristaltic effect, enhancing blood flow from the renal veins towards the heart. This process reduces renal vein pressure and improves renal perfusion, ultimately enhancing patient outcomes. Further examples may include more than three balloons to enable a peristaltic pumping effect, placed on an elongate member such as a catheter in the inferior vena cava.

In some examples, the system comprises four main balloons:
Distal Occlusion Balloon: This balloon is configured to occlude a distal portion of the IVC, creating a controlled environment for the device to operate.
Proximal Occlusion Balloon: Positioned proximally within the IVC, this balloon is configured to occlude the proximal portion of the IVC, working in tandem with the distal occlusion balloon to target the section of the IVC around the renal veins.
Middle Balloon Segment: Located between the distal and proximal occlusion balloons, this balloon inflates and deflates rhythmically to facilitate venous return and reduce renal vein pressure.
Pulmonary Artery Balloon Segment: A smaller balloon catheter is advanced through a distal opening in the distal occlusion balloon into the right atrium, right ventricle, and further into the pulmonary artery. This balloon is positioned to measure pulmonary pressures without acting as a pump.

The three proximal balloons—the distal occlusion balloon, proximal occlusion balloon, and middle balloon segment—are automatically controlled by an external processor. This processor receives real-time data from integrated sensors, which monitor blood pressure at the location of the renal veins and/or the right atrium pressure. Based on this data, the processor dynamically adjusts the inflation and deflation cycles of these balloons to optimize venous return and reduce renal vein pressure.

The Pulmonary Artery Balloon Segment, positioned in the pulmonary artery, includes sensors that measure pulmonary pressures, providing critical data to inform overall cardiovascular management. Although this balloon does not act as a pump, it plays a vital role in monitoring pulmonary artery conditions, ensuring comprehensive hemodynamic monitoring.

The automated control system for the lower three balloons involves precise timing and coordination to match the physiological needs of the patient. By inflating and deflating these balloons in a synchronized manner, the system effectively regulates IVC pressure, enhances venous return, and reduces the load on the renal veins, thereby improving renal function and overall cardiovascular stability.

By combining automated control of the three lower balloons with real-time monitoring of pulmonary pressures, the venous balloon pump system offers a robust solution for managing complex cardiovascular conditions. This system not only reduces renal vein pressure but also provides valuable insights into pulmonary hemodynamics, enhancing patient care and outcomes.

This system not only helps in optimizing volume status and guiding diuretic therapy but also plays a crucial role in identifying areas of ischemia and guiding the administration of medications such as vasopressors. The primary function of the middle balloon is to rhythmically inflate and deflate, creating a pulsatile flow that facilitates venous return and reduces pressure within the renal veins.

Anatomically, the IVC in adults typically ranges from 20 to 30 mm in diameter. To ensure effective occlusion and pressure modulation, the middle balloon is designed to expand up to a diameter of 20-30 mm when inflated. The balloon length, spanning approximately 40-70 mm (4-7 cm), is sufficient to create a significant occlusive effect, reduce renal vein pressure, and maintain adequate blood return to the heart.

The frequency of the balloon's inflation and deflation cycles is crucial for maintaining continuous blood flow and effective pressure reduction. To achieve this, the balloon is designed to pump at a frequency that matches or slightly exceeds the heart rate, typically 60-120 times per minute. This synchronization ensures that the balloon action is in harmony with the body's natural circulatory rhythms.

In some examples, the systems and methods may integrate real-time monitoring and control systems, utilizing sensors and impedance catheters to measure venous pressures and cardiac output. This allows for dynamic adjustments to the device operation, ensuring optimal performance and patient safety.

The system also includes a secondary catheter with a smaller balloon and impedance sensors that can be advanced into the pulmonary artery, similar to a Swan-Ganz catheter, providing comprehensive hemodynamic monitoring.

In some examples, the system can induce “mechanical diuresis” by creating a pressure gradient in the IVC, moving extraneous and congesting fluid to high-capacitance vessels below the device. This reduces intracardiac pressures and enhances cardiac output, similar to the effects of diuretics, but through a mechanical approach.

Additionally, the system is designed to manage patient symptoms and improve outcomes by optimizing heart rate and intracardiac pressures. By adjusting both heart rate and the pressure gradient within the IVC, the device helps maintain optimal cardiac output and reduce symptoms of heart failure, such as fatigue and shortness of breath. The system's ability to monitor and respond to changes in hemodynamic parameters ensures that patients receive tailored, real-time adjustments to their treatment, improving overall quality of life.

In some examples, pressure sensors can be placed in the pulmonary artery to monitor hemodynamic changes, providing critical data on pulmonary pressures and helping to assess overall cardiovascular function. In other examples, pressure sensors can be placed in the right atrium to provide real-time pressure measurements, which are essential for understanding right atrial pressure and its fluctuations during different physiological states. Additionally, pressure sensors can be positioned in the superior vena cava to detect venous pressure, allowing for monitoring of central venous pressure, which is an important indicator of cardiac function and fluid status. In some examples, pressure sensors can be placed in the inferior vena cava to monitor pressure changes, providing insights into venous return and identifying potential obstructions or pressure gradients. Pressure sensors can also be placed in the right ventricle to assess ventricular pressures, which is crucial for evaluating right ventricular function and detecting potential issues such as pulmonary hypertension. In some cases, pressure sensors can be placed in the left atrium to monitor left-sided filling pressures, helping to manage conditions like heart failure by providing data on left atrial pressure and its impact on pulmonary circulation. Additionally, pressure sensors can be placed in the coronary sinus to estimate left ventricular pressure, offering a less invasive method to assess left ventricular performance and detect heart failure progression.

Impedance catheters can be used in some examples to measure changes in blood volume, providing continuous data on intravascular volume status and helping to guide fluid management in critically ill patients. In other examples, impedance catheters can be used to measure cardiac output by assessing impedance changes, offering a non-invasive method to monitor cardiac function and adjust treatment plans accordingly. Flow sensors can utilize thermal dilution methods to measure blood flow by injecting a known temperature saline and monitoring temperature changes downstream, providing accurate measurements of cardiac output. Alternatively, dye dilution methods can be used by injecting a known dye and monitoring its concentration downstream, offering another technique to measure blood flow and cardiac output accurately. Electromagnetic flow sensors can measure blood flow by detecting voltage changes in a magnetic field, providing real-time data on flow rates and vascular health. Ultrasound Doppler sensors can measure blood flow velocity using the Doppler effect, offering a non-invasive way to monitor blood flow and detect abnormalities.

In some examples, light oximetry can be used to measure blood oxygen levels using optical sensors, providing continuous monitoring of oxygen saturation, which is critical in managing respiratory and cardiovascular conditions. Fiber optic sensors can detect blood composition such as lactate, pH, and oxygen levels, offering comprehensive data on metabolic and respiratory status. Electrode-based sensors can measure electrical impedance related to blood volume changes, providing insights into fluid status and cardiac function. They can also be used for electrophysiological mapping of the heart, identifying arrhythmias and guiding ablation therapies. Ultrasonic transducers can be used for real-time imaging and flow measurement, providing detailed images of blood vessels and flow dynamics. They can also identify venous vasculature for optimal catheter placement, ensuring accurate and safe interventions. Piezoelectric sensors can detect vessel wall vibrations associated with blood flow, providing data on flow characteristics and vessel health. Micro-electromechanical systems (MEMS) sensors can detect changes in fluid dynamics to measure blood flow, offering highly sensitive and precise measurements. Digital image analysis can use embedded micro-cameras to capture real-time images of blood flow for flow rate measurement, providing detailed visual information to support clinical decision-making.

In one example, a differential pressure method is provided, which utilizes sensors on either side of an inflated balloon or vessel obstruction to measure the pressure differential, which is directly related to flow rate according to Bernoulli's principle. Other examples include thermal dilution or dye dilution, which injects a known volume and temperature of saline or a known quantity of biocompatible dye upstream and monitors the dye concentration downstream using optical sensors, or measures the change in temperature downstream after mixing with blood, using a thermistor or other temperature measurement device. The flow rate is calculated based on the dilution of the saline's temperature or dilution of the dye. Some examples may include heating or cooling the blood flow by means of other fluid or direct heating and cooling from the catheter.

Another example uses an ultrasound Doppler effect embedded on the balloon or obstruction device or on the catheter to determine the flow rate. In this example, one or more miniature ultrasonic transducers are embedded within the catheter wall or on the obstruction device to generate and detect ultrasound waves. The Doppler shift in the reflected waves provides a measure of blood flow rate. These ultrasonic transducers may additionally be used to identify venous vasculature for optimal balloon placement (i.e. by identifying the renal veins from within the inferior vena cava). There may be a plurality of ultrasonic or other sensors surrounding the balloon, and flow around these sensors or other sensors may cleanse them and prevent build-up.

Sensors in this example and others may be used to identify the volume status of a patient. In some examples, light sensors may be used to detect lactate, pH levels, oxygen, and other parameters of the blood. Placement of the sensor at the balloon or obstruction may utilize blood flow to prevent thrombus or loss in light degradation. Electrodes on the balloon catheter, catheter, or obstruction device may be used for impedance-based mapping to determine vessel size, location within the vessel, and optimal placement without necessitating use of fluoroscopy. Alternatively or in addition, the device leverages fiber optic technology and specialized coatings to provide real-time, accurate readings of blood composition. A fiber optic strand may be coated with a reactive material or dye that can produce a detectable optical change in response to specific blood parameters, such as oxygen saturation, carbon dioxide and pH measurements, hydrogen ion, lactate, coagulation, lipid levels, metabolites, circulating tumor cells, glucose, hemoglobin concentration, bilirubin levels, or others.

Other examples include implementing a miniaturized electromagnetic flowmeter inside the catheter, an optical sensor or fiber, or a micro impeller. For the magnetic flow sensor, as blood flows through the magnetic field, it generates a voltage proportional to its flow rate. The optical sensing technique uses embedded optical fibers to emit and detect light. As blood cells move, they scatter light in a manner that can be analyzed to determine the flow rate. For the micro impellor, the system may incorporate a tiny, rotationally-sensitive element or impeller within or outside of the catheter lumen. The speed at which blood forces this impeller to rotate indicates the flow rate.

Further examples may utilize piezoelectric sensors embedded within or on the catheter or balloon. These sensors detect subtle vibrations or changes in the vessel wall associated with blood flow, which can be converted to flow rate measurements. Similarly, MEMS-type sensors incorporate micro-electromechanical systems (MEMS) within the catheter to detect changes in fluid dynamics and derive blood flow rate. Digital image analysis may be used in a different example, using an embedded micro-camera to capture real-time images of the blood flow. These images are then digitally processed to ascertain the flow rate.

In one example, the system may comprise four main components:
Distal Occlusion Balloon: This balloon is configured to occlude a distal portion of the IVC, creating a controlled environment for the device to operate.
Proximal Occlusion Balloon: Positioned proximally within the IVC, this balloon is configured to occlude the proximal portion of the IVC, working in tandem with the distal occlusion balloon to target the section of the IVC around the renal veins.
Middle Balloon Segment: Located between the distal and proximal occlusion balloons, this balloon inflates and deflates rhythmically to facilitate venous return and reduce renal vein pressure.
Adjustable Pulmonary Artery Balloon Catheter: A smaller balloon catheter that can be advanced or withdrawn through a distal opening in the distal occlusion balloon into the right atrium, right ventricle, and further into the pulmonary artery. This balloon catheter is designed to measure pulmonary pressures and can be precisely positioned as needed.

The three lower balloons—the distal occlusion balloon, proximal occlusion balloon, and middle balloon segment—are automatically controlled by an external processor. This processor receives real-time data from integrated sensors that monitor blood pressure at the location of the renal veins, right atrium pressure, and overall IVC pressure. Based on this data, the processor dynamically adjusts the inflation and deflation cycles of these balloons to optimize venous return and reduce renal vein pressure, ensuring efficient blood flow through the IVC.

The Adjustable Pulmonary Artery Balloon Catheter is designed to provide flexibility in its positioning. It can be advanced or withdrawn as required to obtain optimal measurements of pulmonary pressures. This adjustability allows healthcare providers to position the balloon catheter precisely within the pulmonary artery, right ventricle, or right atrium, depending on the specific diagnostic or therapeutic needs. The capability to advance or withdraw this catheter enhances the versatility of the system, enabling it to adapt to changing clinical conditions and providing detailed hemodynamic data from different cardiac and pulmonary locations.

Some examples of the system may involve targeted energy delivery to administer treatment, including: Electroporation Mechanism: Electroporation involves applying short, high-voltage electrical pulses to cells, which creates temporary pores in cell membranes. This can be used to disrupt bacterial cells, leading to their destruction, or to enhance the uptake of antibiotics into bacterial cells, making the treatment more effective. Energy Delivery: Other forms of energy, such as ultrasound or photodynamic therapy, could also be integrated into the device to target bacteria. These methods can disrupt bacterial cell walls, enhance antibiotic action, or directly kill pathogens. Electroporation Catheter: The system could include an additional catheter equipped with electrodes that can be advanced through the existing catheter system into the bloodstream. This catheter would deliver precise electrical pulses to the targeted area. Energy Control Unit: A control unit integrated with the processor could manage the delivery of electrical pulses or other energy forms, ensuring that the application is precise and effective. The control unit would also monitor energy levels to prevent damage to surrounding tissues. Bacterial Destruction: Delivering electroporation or other energy forms directly to the bloodstream can effectively kill circulating bacteria, reducing the overall bacterial load and aiding in faster infection control. Enhanced Antibiotic Efficacy: Electroporation can increase the permeability of bacterial cell membranes, allowing antibiotics to enter more easily and work more effectively. This can be particularly useful in combating antibiotic-resistant bacteria. Localized Treatment: Targeting specific areas with energy delivery minimizes systemic side effects and focuses the treatment where it is most needed, such as in regions identified with high bacterial load or poor antibiotic penetration.

Various nerve plexi can be targeted by the system to improve patient outcomes. These include: Cardiac Plexus: Located around the base of the heart, particularly near the aortic arch and the pulmonary artery. The cardiac plexus supplies autonomic innervation to the heart, influencing heart rate, force of contraction, and coronary blood flow. A balloon catheter in the heart or ascending aorta could stimulate the cardiac plexus, potentially modulating heart rate and myocardial contractility through electrical or mechanical stimulation. Pulmonary Plexus: Situated around the roots of the lungs and along the trachea and bronchi. The pulmonary plexus provides autonomic innervation to the lungs, regulating bronchoconstriction, bronchodilation, and mucus secretion. A catheter placed in the superior vena cava (SVC) near the heart could influence the pulmonary plexus to modulate respiratory functions. Celiac (Solar) Plexus: Positioned around the celiac artery, just below the diaphragm near the IVC. The celiac plexus supplies autonomic innervation to the abdominal organs, including the stomach, liver, pancreas, kidneys, and intestines. A balloon catheter in the IVC could potentially target the celiac plexus to influence digestive and renal functions, as well as modulate pain responses in the upper abdomen. Renal Plexus: Surrounding the renal arteries and extending along the renal veins into the kidneys. The renal plexus provides autonomic innervation to the kidneys, influencing renal blood flow, glomerular filtration rate, and renin release. A balloon catheter in the IVC near the renal veins could target the renal plexus to modulate renal function and blood pressure regulation. Aortic Plexus: Along the abdominal aorta, extending from the thoracic aorta down to the bifurcation of the aorta. The aortic plexus provides autonomic innervation to the abdominal aorta and its branches, affecting blood flow distribution and vessel tone. A catheter in the IVC or abdominal aorta could influence the aortic plexus to modulate vascular resistance and blood pressure. Superior Hypogastric Plexus: Situated in front of the lower part of the aorta and the bifurcation of the common iliac arteries. The superior hypogastric plexus supplies autonomic innervation to the pelvic organs, including the bladder, rectum, and reproductive organs. A balloon catheter in the IVC near the bifurcation could influence the superior hypogastric plexus to modulate pelvic organ functions.

This and other examples may include: Indirect Electrical Stimulation: While the vagus nerve itself is not adjacent to the IVC, indirect modulation of the autonomic system can be achieved by targeting other nerves or structures influenced by autonomic activity. This could involve stimulating the IVC's surrounding tissues or adjacent structures that have autonomic connections. This could include nerves within the autonomic plexuses that are more accessible from the IVC. For example, the celiac plexus, which has autonomic components and lies near the IVC in the abdominal cavity, could be a potential target. Pharmacological Modulation: The catheter can be used to deliver pharmacological agents that modulate the autonomic nervous system. These agents could include drugs that mimic or block autonomic neurotransmitters. Agents such as acetylcholine receptor agonists/antagonists or adrenergic receptor agonists/antagonists can be infused to influence autonomic tone. Reflex Modulation: Stimulation of baroreceptors or chemoreceptors, which are part of the autonomic feedback mechanisms, can modulate autonomic activity. These receptors can influence heart rate, blood pressure, and respiratory rate. The catheter could be used to apply mild pressure or electrical stimulation to areas rich in these receptors. For instance, the renal nerves, which have significant autonomic innervation, could be targeted indirectly by altering renal perfusion and triggering renal baroreceptors.

Potential Methods to Implement Autonomic Modulation in the IVC Catheter may include: Electrical Stimulation via Adjacent Structures: Use electrodes integrated into the catheter to deliver electrical pulses to the IVC wall. While the IVC itself may not directly stimulate autonomic nerves, the electrical fields could influence nearby autonomic plexuses. Electrical fields can be adjusted to target regions where autonomic nerves are more likely to be present, such as near the adrenal glands or renal arteries. Localized Drug Delivery: Administer drugs that modulate autonomic activity through the catheter. Drugs could be delivered in a controlled manner to specific areas around the IVC. Parasympathomimetics (e.g., pilocarpine) or sympathomimetics (e.g., norepinephrine) could be used to enhance or suppress autonomic activity. Thermal Modulation: Applying localized cooling or heating through the catheter to influence autonomic nerves. Thermal modulation can alter nerve activity by changing the temperature of surrounding tissues. Localized heating could enhance sympathetic activity, while cooling could reduce it. Mechanical Stimulation: Utilize the catheter to apply gentle mechanical pressure to the IVC or surrounding tissues to influence autonomic reflexes. Controlled inflation of balloons within the IVC to apply intermittent pressure, potentially triggering autonomic reflexes mediated by nearby structures. Direct pressure from the balloon or indirectly by increasing blood venous can be utilized.

In some examples, the system can be used to oxygenate the blood. The pumping function of the venous balloon pump system can indeed play a significant role in improving oxygenation by increasing blood flow and enhancing the mixing of blood, which can optimize gas exchange in the integrated oxygenator.

Enhanced Blood Flow and Mixing: The rhythmic inflation and deflation of the middle balloon segment create a pulsatile flow that enhances the mixing of venous blood. This increased turbulence and mixing can improve the efficiency of gas exchange by exposing more blood to the gas-permeable fibers or membranes. Enhanced mixing ensures that more red blood cells come into contact with the oxygenation surfaces, increasing the overall oxygen content of the blood and promoting more uniform oxygenation.

Driving Blood Through the Oxygenator: The increased pressure generated by the balloon pump can be harnessed to drive blood through the gas-permeable fibers or membranes within the oxygenator. This can be achieved by integrating the oxygenator directly into the catheter system, where the pumping action ensures a continuous flow of blood through the oxygenation module. By actively pumping blood through the oxygenator, the system can maintain a steady and controlled blood flow, optimizing the conditions for gas exchange and ensuring efficient oxygenation and carbon dioxide removal.

Pressure-Driven Oxygenation: The increased intravascular pressure from the balloon inflation can be used to enhance the diffusion of gases across the membranes. Higher pressure gradients can improve the rate of oxygen diffusion into the blood and the removal of carbon dioxide. Pressure-driven oxygenation can increase the efficiency of gas exchange, particularly in scenarios where oxygen demand is high.

Some examples of this system include: Integrated Oxygenation Module: The oxygenation module can be integrated into the catheter system, where blood flows through gas-permeable fibers or membranes positioned along the catheter. The pumping action ensures continuous blood flow through this module. The oxygenation module can be placed proximally to the middle balloon segment, utilizing the pressure generated by the balloon to drive blood through the module. Enhanced Mixing Design: The design of the catheter and oxygenation module can include features that enhance blood mixing, such as helical flow channels or turbulence-inducing structures. These design features can improve the contact between blood and the gas-permeable surfaces, enhancing the efficiency of oxygenation.

In other examples, the venous balloon pump system incorporates a novel distal gas exchange segment designed to efficiently remove carbon dioxide (CO2) and potentially deliver oxygen (O2) directly within the inferior vena cava (IVC). This segment utilizes hollow fiber membranes or other gas-permeable materials to facilitate gas exchange, providing critical respiratory support to patients with severe respiratory and circulatory challenges.

Positioning and Deployment: The catheter is introduced into the IVC through a peripheral vein, typically via a minimally invasive procedure. The distal gas exchange segment is positioned within the distal IVC. Once correctly positioned, the system is activated to deploy the gas exchange membranes. This can be achieved by shortening the catheter, which forces the hollow fiber membranes to expand outward, or by spinning the segment, which unwraps and expands the membranes.

Gas Exchange Process: Blood flowing through the IVC passes over the gas-permeable hollow fiber membranes. CO2 from the blood diffuses across the membrane walls into the sweep gas channel, which carries the CO2 away from the bloodstream. If needed, oxygen can be delivered into the blood through the same hollow fiber membranes. Oxygen diffuses from the sweep gas into the blood, enhancing the oxygen content and supporting tissue oxygenation.

Shortening Mechanism: The distal segment consists of a bundle of hollow fiber membranes arranged longitudinally along the catheter. Upon activation, the catheter is shortened slightly, causing the hollow fibers to compress and expand outward. This expansion increases the surface area in contact with the blood, enhancing the efficiency of gas exchange. The expanded membranes partially occlude the IVC, ensuring that a significant portion of the blood flow contacts the gas exchange surfaces, maximizing CO2 removal and O2 delivery.

Spinning Mechanism: The hollow fiber membranes are wrapped around the distal segment of the catheter. When the segment is spun, the wrapped membranes unwrap and expand outward into the IVC. This spinning action ensures even distribution of the membranes, creating a large surface area for gas exchange. The expanded membranes create a partial occlusion of the IVC, directing blood flow over the gas-permeable surfaces for efficient gas exchange.

This and other examples may comprise these System Components and Operation: Hollow Fiber Membranes: Made from biocompatible, gas-permeable materials such as silicone or polypropylene, designed to allow efficient diffusion of gases. They will facilitate the exchange of CO2 and O2 between the blood and the sweep gas. Sweep Gas System: Inlet and Outlet Channels are integrated within the catheter, providing a continuous flow of sweep gas (air or a specific gas mixture) through the hollow fibers. The sweep gas carries CO2 away from the membranes, preventing reabsorption into the bloodstream. The same sweep gas can deliver oxygen into the blood if needed, enhancing oxygenation. Control and Monitoring: A Processor regulates the deployment of the gas exchange segment, the flow of sweep gas, and the pumping action of the middle balloon. Sensors monitor blood pressure, flow rates, CO2 and O2 levels, ensuring optimal performance and safety.

Beyond shortening and spinning mechanisms, there are several innovative ways to control the expansion of hollow fiber membranes within the inferior vena cava (IVC) to facilitate gas exchange. These mechanisms ensure that the membranes deploy effectively, maximizing contact with blood flow for efficient CO2 removal and O2 delivery.

Balloon-Assisted Expansion: Small inflatable balloons are integrated along the length of the distal segment of the catheter. When the balloons are inflated, they push the hollow fiber membranes outward into the blood flow within the IVC. This provides precise control over the expansion and can be adjusted dynamically based on the patient's needs. The catheter is inserted into the IVC with the balloons deflated. Upon reaching the desired position, the balloons are gradually inflated, expanding the membranes outward. The degree of expansion can be fine-tuned by adjusting the balloon inflation pressure.

Radial Expansion Mechanism: The distal segment includes a series of radial expanders made from a flexible but firm material. These expanders are deployed outward, pushing the hollow fiber membranes into the blood flow. This provides uniform expansion and maintains a stable structure within the IVC. The radial expanders are initially compressed within the catheter. A control mechanism (e.g., a pull wire or hydraulic system) is activated to expand the radial structures outward. Once deployed, the radial expanders hold the hollow fibers in place, ensuring consistent contact with blood flow.

Self-Expanding Stent-Like Structure: The distal segment includes a self-expanding stent-like mesh structure that supports the hollow fiber membranes. The mesh structure expands automatically once released from the catheter sheath. This is a simple deployment process and robust expansion, ensuring high surface area contact. The catheter is inserted into the IVC with the mesh structure compressed within a sheath. When the sheath is retracted, the mesh structure self-expands, deploying the hollow fibers. The expanded mesh provides a stable framework for the membranes.

Shape Memory Alloys: The distal segment incorporates Shape Memory Alloy (SMA) elements that change shape in response to temperature or electrical stimuli. These elements expand the hollow fiber membranes when activated. This provides precise control and can be repeatedly actuated. The catheter is placed in the IVC with the SMA elements in their compressed state. Applying heat or an electrical current activates the SMA elements, causing them to expand and deploy the membranes. The SMA elements can revert to their original shape if deactivated, allowing for controlled deployment and retraction.

Mechanical Deployment with Pull Wires: The distal segment includes pull wires that mechanically deploy the hollow fiber membranes. Pulling on the wires expands the membranes outward. This provides direct mechanical control over the expansion process. The catheter is positioned within the IVC with the membranes in a compressed state. Pulling on the wires expands the membranes, positioning them into the blood flow. The expansion can be precisely controlled by adjusting the tension on the pull wires.

Expandable Mesh Design: Create an expandable mesh structure using hollow fibers, which can be collapsed for insertion and then expanded within the IVC to occlude blood flow. The mesh can be designed to expand radially to occlude the IVC and ensure the fibers are appropriately oriented for optimal gas exchange.

Balloon-Stent Hybrid: Integrate the hollow fibers into a balloon-stent hybrid device. The stent can be expanded to anchor the device in place, and the balloon can be inflated to ensure the fibers facilitate gas exchange. This design would allow controlled expansion and ensure the fibers remain in place and effective for gas exchange.

Helical Coil Structure: Design the hollow fibers in a helical coil that can expand and contract. The coil can be compact during insertion and then expanded to occlude the IVC. The helical structure can provide flexibility and a large surface area for gas exchange while maintaining mechanical stability.

Umbrella-Type Occluder: Use an umbrella-type occluder design where the hollow fibers are arranged in a radial pattern that expands like an umbrella to occlude the IVC. This design would ensure complete occlusion and provide a large surface area for gas exchange within the IVC.

Incorporating hollow fiber membranes for CO2 removal and O2 delivery into the distal segment of a venous balloon pump catheter involves innovative expansion mechanisms to ensure efficient gas exchange within the IVC. Mechanisms such as balloon-assisted expansion, radial expanders, self-expanding stent-like structures, shape memory alloys, and mechanical pull wires provide various methods to deploy and stabilize the hollow fibers effectively.

Hollow fiber membranes used in gas exchange devices, such as those incorporated into a venous balloon pump system for CO2 removal and O2 delivery, must possess specific properties to ensure efficient and safe gas exchange. These properties include high gas permeability, biocompatibility, mechanical strength, and resistance to fouling. Here are some potential materials commonly used for gas-permeable membranes:

Polymers: Polypropylene (PP) has high gas permeability, good mechanical strength, and chemical resistance, and is widely used in hollow fiber membranes due to its durability and ease of fabrication. Polysulfone (PSU) has excellent thermal stability, high mechanical strength, and good chemical resistance and provides a balance between performance and durability, commonly used in medical applications. Polyethersulfone (PES) has high permeability, excellent thermal and chemical resistance, and good mechanical properties and is used in a variety of medical and industrial filtration applications due to its robust properties. Silicone has exceptional biocompatibility, flexibility, and high gas permeability and is ideal for medical applications requiring direct contact with blood and tissues. Polyvinylidene Fluoride (PVDF) has high chemical resistance, good mechanical strength, and high gas permeability and is commonly used in filtration and gas exchange applications due to its stable properties. Polydimethylsiloxane (PDMS) has high gas permeability, flexibility, and biocompatibility and is an excellent material for applications requiring high gas transfer rates and flexibility. Polymethylpentene (PMP) has high gas permeability, good chemical resistance, and excellent clarity and is known for its high oxygen permeability, making it suitable for applications in oxygenators and other gas exchange devices.

Composite Materials: Microporous PTFE (Polytetrafluoroethylene) has exceptional chemical resistance, high gas permeability, and biocompatibility and is used in applications requiring durable and chemically inert membranes. Polyolefin Blends utilizes a combination of different polyolefins to enhance gas permeability and mechanical properties and can have tailored properties to suit specific gas exchange needs. Cellulose Acetate has good biocompatibility, moderate gas permeability, and hydrophilicity and is used in dialysis membranes and other medical applications.

Advanced and Hybrid Materials: Carbon Nanotubes (CNTs) have high mechanical strength, excellent thermal and electrical conductivity, and high gas permeability and the potential for high-performance membranes with enhanced gas transfer rates. Graphene Oxide has high surface area, excellent mechanical properties, and high gas permeability and is an emerging material with potential for advanced gas exchange applications. Metal-Organic Frameworks (MOFs have high porosity, tunable structures, and exceptional gas adsorption properties and are innovative materials for enhanced gas separation and exchange efficiency. Zeolite has high thermal stability, high surface area, and selective gas permeability and is used in advanced gas separation applications due to its unique porous structure.

In some examples, the venous balloon pump has a balloon pumping agent and proximal and distal occlusion segments. The proximal and distal occlusion segments may include hollow fiber members that can be enlarged within the vena cava to occlude blood flow to create an area of low pressure as well as help gas exchange.

In some examples, the venous balloon pump system is equipped with a balloon pumping agent and proximal and distal occlusion segments. The primary function of these occlusion segments is to manage blood flow within the vena cava, creating an area of low pressure that facilitates gas exchange. These occlusion segments are designed to include hollow fiber members, which can be expanded to occlude blood flow effectively.

Proximal and Distal Occlusion Segments: The proximal and distal occlusion segments are strategically positioned within the inferior vena cava (IVC). Each segment consists of hollow fiber members that, when expanded, create a partial or complete occlusion of blood flow. This occlusion serves a dual purpose: it generates an area of low pressure conducive to gas exchange and supports the mechanical action of the balloon pumping agent. The occlusion segments ensure that blood is directed through the gas exchange fibers, enhancing the efficiency of CO2 removal and O2 delivery.

Hollow Fiber Members and Gas Exchange: The hollow fiber members embedded within the occlusion segments are made of gas-permeable materials, such as polypropylene, polysulfone, or polymethylpentene. These materials allow for efficient diffusion of gases across their membranes. When the fibers are enlarged within the vena cava, they create a surface area that facilitates the transfer of gases. CO2 from the blood diffuses into the hollow fibers and is carried away by a sweep gas, while O2 can be delivered from the sweep gas into the bloodstream if needed.

Low Pressure Area and Enhanced Efficiency: By occluding blood flow and creating an area of low pressure, the system optimizes conditions for gas exchange. The low-pressure zone ensures that blood remains in contact with the gas-permeable membranes for a sufficient duration, allowing for maximal CO2 removal and O2 delivery. The balloon pumping agent, synchronized with the patient's physiological parameters, drives blood through the occlusion segments, ensuring continuous and efficient gas exchange. This innovative design enhances the overall functionality of the venous balloon pump system, making it an effective solution for managing respiratory and circulatory challenges in critically ill patients.

Clinical Applications: The integration of hollow fiber members in the occlusion segments of the venous balloon pump system offers significant advantages in clinical settings. For patients in septic shock or those undergoing high-risk surgeries, the system provides crucial respiratory support by efficiently removing CO2 and delivering O2. The creation of a low-pressure area within the vena cava enhances gas exchange efficiency, improving patient outcomes and reducing the burden on traditional respiratory support systems. This advanced design represents a significant advancement in the management of respiratory and circulatory conditions, offering a versatile and effective tool for critical care.

The balloon catheter may be placed in the superior vena cava, the inferior vena cava, and the pulmonary artery, among other places for both pumping of blood and gas exchange.

In yet another example, a pump is used to pump the blood, instead of an expanding and collapsing balloon. These pumps may be centrifugal or axially pumps. Centrifugal pumps use a rotating impeller to generate a centrifugal force that propels blood radially outward from the center of the impeller to the periphery. As the impeller spins, it creates a low-pressure area at the center, drawing blood into the pump and then forcing it outwards through the outlet, creating continuous flow.

Axial flow pumps use a helical screw or impeller that moves blood along the axis of the pump, creating a continuous flow along the length of the pump. The rotating screw or impeller generates a continuous forward motion, pushing blood from the inlet to the outlet in a smooth and controlled manner.

The venous balloon pump system is designed to incorporate a pump mechanism, a proximal occlusion agent, and a distal segment specifically designed to facilitate gas exchange. This system is tailored to provide efficient respiratory support by removing carbon dioxide (CO2) and delivering oxygen (O2) directly within the inferior vena cava (IVC). Additionally, the pump creates an area of low pressure around the renal veins to improve renal function.

System Configuration: Pump Mechanism: The system includes a robust pump designed to drive blood flow through the catheter system. This pump ensures continuous circulation of blood and maintains optimal flow rates for effective gas exchange. Integrated with the pump is an occlusion or anchoring mechanism that prevents backflow around the pump, ensuring unidirectional blood flow and enhancing the system's efficiency. The pump is specifically designed to create an area of low pressure around the renal veins, which can improve renal function by enhancing blood flow and reducing venous congestion. Proximal Occlusion Agent: The proximal occlusion agent is positioned within the IVC, near the renal veins. This segment is designed to partially occlude the IVC, creating an area of low pressure that enhances the dynamics of blood flow and promotes effective gas exchange. The proximal occlusion agent can be composed of various biocompatible materials that ensure a secure fit within the IVC and help direct blood flow towards the distal gas exchange segment. Distal Gas Exchange Segment: The distal segment of the catheter is equipped with hollow fiber membranes made from gas-permeable materials such as polypropylene, polysulfone, or polymethylpentene. This segment is responsible for facilitating gas exchange by allowing CO2 to diffuse out of the blood and O2 to diffuse into the blood. The hollow fibers expand within the IVC to maximize the surface area in contact with the blood flow.

Mechanism of Action: Blood Flow and Pump Operation: The pump mechanism drives blood through the catheter system, ensuring continuous circulation. The pump is equipped with an occlusion or anchoring mechanism to prevent backflow and maintain unidirectional flow. The pump's operation is controlled by a processor that adjusts the flow rate based on real-time physiological data, ensuring optimal conditions for gas exchange and renal function improvement. Proximal Occlusion and Low-Pressure Area: The proximal occlusion agent expands within the IVC, creating a partial occlusion that generates a localized area of low pressure near the renal veins. This low-pressure area directs blood flow towards the distal gas exchange segment, enhancing the efficiency of CO2 removal and O2 delivery. Additionally, the reduced pressure around the renal veins improves renal function by decreasing venous congestion and enhancing blood flow through the kidneys. Gas Exchange Process in the Distal Segment: As blood flows through the distal gas exchange segment, CO2 diffuses across the hollow fiber membranes into the sweep gas channel, where it is carried away. Oxygen from the sweep gas diffuses into the blood through the same hollow fibers, increasing the oxygen content of the blood. The hollow fiber membranes in the distal segment are designed to expand and conform to the inner walls of the IVC, maximizing contact with the blood flow.

Enhanced Surface Area for Gas Exchange: Hollow Fiber Membrane Arrangement: The hollow fiber membranes are densely packed within the distal segment, significantly increasing the surface area available for gas exchange. The enhanced design allows the distal segment to achieve a substantial surface area, potentially up to 0.33 sqm per segment, facilitating effective gas exchange. Stability and Efficiency: The occlusion or anchoring portion of the pump stabilizes the catheter within the IVC, ensuring that the proximal occlusion agent and distal gas exchange segment remain securely in place. The pump mechanism maintains continuous blood flow through the occlusion agent and gas exchange segment, optimizing contact time with the gas-permeable membranes for efficient gas exchange. Pump Mechanism: The system includes a robust pump designed to drive blood flow through the catheter system. This pump ensures continuous circulation of blood and maintains optimal flow rates for effective gas exchange. Occlusion or Anchoring Portion: Integrated with the pump is an occlusion or anchoring mechanism that prevents backflow around the pump, ensuring unidirectional blood flow and enhancing the system's efficiency. The pump is specifically designed to create an area of low pressure around the renal veins, which can improve renal function by enhancing blood flow and reducing venous congestion. Proximal Occlusion Agent: The proximal occlusion agent is positioned within the IVC, near the renal veins. This segment is designed to partially occlude the IVC, creating an area of low pressure that enhances the dynamics of blood flow and promotes effective gas exchange. The proximal occlusion agent can be composed of various biocompatible materials that ensure a secure fit within the IVC and help direct blood flow towards the distal gas exchange segment. Distal Gas Exchange Segment: The distal segment of the catheter is equipped with hollow fiber membranes made from gas-permeable materials such as polypropylene, polysulfone, or polymethylpentene. This segment is responsible for facilitating gas exchange by allowing CO2 to diffuse out of the blood and O2 to diffuse into the blood. The hollow fibers expand within the IVC to maximize the surface area in contact with the blood flow.

Concept of Using Electroporation Energy to Facilitate Gas Exchange: Electroporation Overview: Electroporation is a technique that utilizes short, high-intensity electrical pulses to create temporary pores in cell membranes. This method is widely used in various biomedical applications, including gene therapy, drug delivery, and tissue ablation. The technology operates by applying pulsed electric fields to cells, which disrupts the lipid bilayer of cell membranes, thereby increasing their permeability. Electroporation in Gas Exchange: In the context of a venous balloon pump system, electroporation energy can be employed to enhance gas exchange within the blood. The principle here is similar to that used in pulsed field ablation (PFA) technology, which delivers controlled electrical pulses to target tissues. By applying electroporation energy to the blood, the permeability of red blood cells and other blood components can be transiently increased. This enhanced permeability facilitates the more efficient release of carbon dioxide (CO2) and the absorption of oxygen (O2) across cell membranes. Mechanism of Action: The catheter system integrates an electroporation unit that delivers pulsed electric fields to the blood as it flows through the distal and proximal occlusion segments. The electrical pulses induce electroporation in blood cells, temporarily creating pores in their membranes. The temporary pores allow for more rapid and efficient diffusion of gases. CO2, which is primarily transported in the blood as bicarbonate and dissolved gas, can more easily diffuse out of the red blood cells and plasma into the hollow fiber membranes of the gas exchange segments. Similarly, O2 can diffuse more readily into the red blood cells, enhancing oxygenation.

Additional Benefits may include: Bacterial Inactivation: Beyond facilitating gas exchange, the application of electroporation energy has the potential to kill bacteria present in the blood. The electrical pulses can disrupt bacterial cell membranes, leading to cell lysis and death. This antimicrobial effect can be particularly beneficial in septic patients, where bacterial contamination of the blood is a significant concern. Improved Circulatory Support: By enhancing the efficiency of gas exchange, electroporation can improve the overall efficacy of the venous balloon pump system. Patients with compromised respiratory function can benefit from more effective CO2 removal and O2 delivery, which are critical for maintaining acid-base balance and ensuring adequate tissue oxygenation.

Implementation in the Venous Balloon Pump System: The venous balloon pump system equipped with electroporation capability comprises several key components: Electroporation Unit: Integrated within the catheter, this unit generates and delivers pulsed electric fields to the blood as it flows through the gas exchange segments. Hollow Fiber Membranes: These membranes, located in the proximal and distal occlusion segments, facilitate the actual gas exchange process. The enhanced permeability of blood cells due to electroporation ensures maximum efficiency of these membranes. Sensors and Processor: The system includes sensors to monitor blood pressure and gas concentrations. The processor uses this data to control the electroporation pulses, ensuring they are delivered at optimal times and intensities for effective gas exchange and bacterial inactivation.

It will be appreciated that elements or components shown with any example herein are exemplary for the specific example and may be used on or in combination with other examples disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. A venous balloon pump system for reducing renal vein pressure, comprising:
an elongate catheter comprising a distal portion sized for introduction into an inferior vena cava of a patient and comprising at least three balloon segments:
a) a distal balloon segment configured to occlude a distal portion of the inferior vena cava upon inflation;
b) a proximal balloon segment configured to occlude a proximal portion of the inferior vena cava upon inflation;
c) a middle balloon segment located between the distal and proximal balloon segments, the middle balloon configured to inflate and deflate to facilitate blood flow and reduce renal vein pressure;
a first sensor configured to monitor blood pressure adjacent the renal veins of the patient;
a second sensor configured to monitor pressure adjacent a right atrium of the patient's heart;
a processor configured to receive data from the first and second sensors and control the inflation and deflation of the middle balloon based at least in part on the monitored blood pressures.

2. The system of claim 1, further comprising a third sensor configured to monitor blood pressure proximal to the proximal balloon segment.

3. The system of claim 2, further comprising:
a fourth balloon segment attached to an elongated member, wherein the elongated member is configured to be advanced or withdrawn through an opening on the distal end of the distal balloon segment, and the fourth balloon segment is configured to be advanced into the pulmonary artery; and
at least one sensor configured to monitor pulmonary artery blood pressure.

4. The system of claim 1, further comprising at least three oxygen sensors configured to monitor oxygen levels in different regions of the patient's body.

5. The system of claim 1, wherein the middle balloon segment is configured to frequently inflate and deflate.

6. The system of claim 5, wherein the middle balloon has a balloon length of at least forty millimeters (40 mm).

7. The system of claim 5, wherein the middle balloon has an inflated diameter of at least twenty millimeters (20 mm).

8. The system of claim 5, further comprising an actuator for inflating and deflating the middle balloon, and wherein the processor is coupled to the actuator to synchronize the frequency of inflation and deflation with the patient's heart rate and/or respiratory rate.

9. The system of claim 1, further comprising a source of inflation media for delivering and removing inflation media from an interior of the middle balloon.

10. The system of claim 9, wherein the source of inflation media comprises a pump and a controller coupled to the pump to control expanding and collapsing the middle balloon.

11. A venous pump system for reducing renal vein pressure or facilitating gas exchange, comprising:
an elongate catheter comprising a distal portion sized for introduction into an inferior vena cava of a patient comprising:
a) a distal occlusion segment configured to occlude a distal portion of the inferior vena cava upon deployment, and further configured with hollow fiber membranes for gas exchange to facilitate the release of carbon dioxide (CO2);
b) a proximal occlusion segment configured to occlude a proximal portion of the inferior vena cava upon deployment;
c) a middle blood pump segment located between the distal and proximal occlusion segments, the middle blood pump segment configured to pump blood and reduce renal vein pressure;
a first sensor configured to monitor blood pressure adjacent renal veins of the patient;
a second sensor configured to monitor pressure adjacent a right atrium of the patient's heart;
a processor configured to receive data from the first and second sensors and control the middle blood pump segment and the distal and proximal occlusion segments based at least in part on the monitored blood pressures.

12. The system of claim 11, wherein the middle blood pump segment comprises a balloon catheter located between the distal and proximal occlusion segments configured to pump blood towards the patient's heart and create a reservoir between the occlusion segments to reduce renal vein pressure.

13. The system of claim 11, wherein the hollow fiber membranes are configured to facilitate adding oxygen to the patient's blood.

14. The system of claim 11, further comprising electroporation unit configured to deliver electroporation energy to the patient's blood, wherein the electroporation energy facilitates gas exchange by increasing the permeability of blood cell membranes, thereby enhancing the release of CO2 and/or the absorption of O2.
